(19) United States
(12) Patent Application Publication
(10) Pub. No.: US 2023/0027472 A1
(43) Pub. Date: Jan. 26, 2023

(54) TARGETING P73 FOR CANCERS RESISTANT TO BH3 MIMETICS

(71) Applicant: BOARD OF REGENTS, THE UNIVERSITY OF TEXAS SYSTEM, Austin, TX (US)

(72) Inventors: Michael ANDREEFF, Houston, TX (US); Yuki NISHIDA, Houston, TX (US); Jo ISHIZAWA, Houston, TX (US)

(21) Appl. No.: 17/773,498

(22) PCT Filed: Oct. 29, 2020
(86) PCT No.: PCT/US20/58066

§ 371 Date: Apr. 29, 2022

Related U.S. Application Data
(60) Provisional application No. 62/928,697, filed on Oct. 31, 2019.

(51) Int. Cl. A61K 45/06; A61K 31/22; A61K 31/496; A61K 31/635; A61K 31/7105; A61P 35/02; A61P 7/00

(52) U.S. Cl. CPC A61K 45/06; A61K 31/22; A61K 31/496; A61K 31/635; A61K 31/7105; A61P 35/02; A61P 7/00

(57) ABSTRACT

Embodiments of the disclosure encompass methods and compositions related to overcoming or preventing cancer resistance to anti-apoptotic proteins, for example BH3 mimetics. The disclosure provides methods for modulating p73 to reduce cancer resistance to BH3 mimetics including inhibitors of Bcl-2, Bcl-xL, Mcl-1, Bcl-W, A1/BFL-1, or a combination thereof. In specific embodiments, targeting isoforms of p73, such as TAp73 or DNp73, results in reduction of cancer resistance to BH3 mimetics to allow effective treatment of hematological cancers.

This application claims priority to U.S. Provisional Patent Application Ser. No. 62/928,697, filed Oct. 31, 2019, which is incorporated by reference herein in its entirety.

### TECHNICAL FIELD

Embodiments of the disclosure include at least the fields of cell biology, molecular biology, and medicine, including cancer medicine.

### BACKGROUND

Recently, BH3 mimetics such as Bcl-2, Bcl-xL, and Mcl-1 inhibitors (e.g., venetoclax, navitoclax) have been introduced for the treatment of hematologic malignancies and cancers. The Bcl-2 inhibitor venetoclax has been FDA-approved for chronic lymphocytic leukemias and acute myeloid leukemias. Clinically, malignancies present or develop resistance to these agents. The present disclosure address a need in the art of providing or enhancing effective BH3 mimetics for cancer treatment.

### BRIEF SUMMARY

The present disclosure is directed to methods and compositions related to cancer therapy, including reducing resistance to a cancer therapy for an individual. Although the cancer may be hematological cancer, in some cases the cancer comprises a solid tumor and may be from any tissue. The cancer resistance to the therapy for the individual may be de novo or it may be acquired after one or more doses of the therapy. In particular embodiments, the resistant cancer is associated with a factor (or multiple factors) identifiable in samples from the individual, and methods of the disclosure concern targeting that factor or factors to modulate it or them, such as inhibit activity and/or production. In specific embodiments, the resistant cancer is associated with the presence of p73 or an isoform thereof, and in certain aspects methods of the disclosure inhibit p73 or an isoform thereof as a direct or indirect mechanism for overcoming the resistance of the cancer.

In particular embodiments, methods of cancer treatment employ one or more BH3 mimetics and one or more agents that target p73 or an isoform thereof, where the two entities may or may not be administered to the individual at the same time or in the same formulation. In specific cases, there are methods of treating an individual for a cancer with one or more BH3 mimetics that includes the step of providing an effective amount of one or more agents that target p73 or an isoform thereof, such as to reduce resistance of the cancer to the BH3 mimetic. Certain embodiments include methods of employing BH3 mimetics that are known to be, or suspected of being, associated with cancer resistance to them, and in specific cases the BH3 mimetics are inhibitors of a protein of the Bcl-2 pathway, such as Bcl-2, Bcl-xL, Mcl-1, Bcl-W, A1/BFL-1, or a mixture thereof. In one specific aspect for an individual, there are methods of overcoming cancer resistance to treatment that comprises both an inhibitor of Bcl-2 in combination with an inhibitor of Bcl-xL, wherein the overcoming of the cancer resistance is related to treatment for the individual with one or more agents that target p73 or an isoform thereof. In another specific aspect for an individual, there are methods of overcoming cancer resistance to treatment with an inhibitor of Bcl-2 in combination with an inhibitor of Mcl-1, wherein the overcoming of the cancer resistance is the result of treatment for the individual of one or more agents that target p73 or an isoform thereof.

Examples of compositions of the disclosure include compositions that comprise one or more BH3 mimetics and that comprise one or more agents that target p73 or an isoform thereof, and in specific cases the compositions further comprise one or more activators of p53. Any of such compositions may be comprised in a pharmaceutically acceptable carrier and including in any formulation suitable for effective administration. Kits comprising any composition of the disclosure are contemplated herein.

Embodiments of the disclosure include compositions comprising: (a) one or more BH3 mimetics; and (b) one or more agents that target p73 or an isoform thereof. In specific embodiments, the composition further comprises (c) one or more activators of p53. The composition may be comprised in a pharmaceutically acceptable excipient. The elements of the compositions of (a) and/or (b) and/or (c) may or may not be housed in separate containers; the elements (a) and/or (b) and/or (c) may be housed in the same container. Any of the compositions may be comprised in a kit or other medical device.

Embodiments of the disclosure include methods of treating cancer, comprising the step of administering to an individual with cancer a therapeutically effective amount of the composition of the disclosure. In specific embodiments, the cancer is resistant to one or more BH3 mimetics. In specific embodiments, in the composition the one or more BH3 mimetics and the one or more agents that target p73 or an isoform thereof are administered to the individual at the same time. In some cases, the one or more BH3 mimetics and the one or more agents that target p73 or an isoform thereof are administered to the individual in the same formulation.

The one or more BH3 mimetics and the one or more agents that target p73 or an isoform thereof may be administered to the individual at different times, in some cases. For example, the one or more BH3 mimetics may be administered to the individual prior to the one or more agents that target p73 or an isoform thereof, in some cases. The one or more BH3 mimetics may be administered to the individual subsequent to the one or more agents that target p73 or an isoform thereof.

Methods of the disclosure may treat any kind of cancer, and the cancer may be a hematological cancer or comprise a solid tumor. Cancers include cancers of the brain, breast, skin, prostate, pancreatic, kidney, bone, spleen, esophagus, stomach, gall bladder, head and neck, cervix, ovary, testes, and so forth. In specific cases, the cancer is chronic lymphocytic leukemia, acute myeloid leukemia, acute lymphocytic leukemia, chronic myeloid leukemia, Hodgkin's lymphoma, non-Hodgkin's lymphoma, or myeloma.

Any p73 isoform may be targeted in methods of the disclosure, including transactivation p73 (Tap73) and/or ΔNp73. In specific cases, the agent that targets p73 or an isoform thereof comprises a small molecule, protein, nucleic acid, or combination or mixture thereof. Examples of small molecules include octyl-(R)-2HG, octyl-(L)-2HG, or a mixture thereof. Examples of nucleic acids include antisense RNAs, shRNA and/or CRISPR molecule. Examples of proteins include antibodies, such as single chain antibodies.

The BH3 mimetic may be of any kind, including one or more inhibitors of Bcl-2, Bcl-xL, Mcl-1, Bcl-W, A1/BFL-1, or a mixture thereof. The BH3 mimetic may be a combination of an inhibitor of Bcl-2 and an inhibitor of Bcl-xL. The BH3 mimetic may be a combination of an inhibitor of Bcl-2 and an inhibitor of Mcl-1. Examples of specific BH3 mimetics include AZD5991, ABT-737, ABT-199 (Venetoclax), ABT-263 (Navitoclax), or a mixture thereof. In certain cases, the BH3 mimetic is not a Bcl-2 inhibitor.

Any individual may be treated with methods and compositions of the disclosure, including of any gender and/or age. In particular embodiments, the subject is a human patient. In specific embodiments, the individual is greater or younger than 1, 5, 10, 20, 30, 40, 50, or 60 years of age. In specific embodiments, the patient is female, while in other embodiments, the patient is male. In other embodiments, the patient is female or male. The patient may be of any gender.

In specific cases of the methods of the disclosure, they may further comprise the step of administering to the individual one or more activators of p53, such as Nutlin-3a, RG7112, RG7388, JNJ-26854165, MI-773, KRT232 (AMG 232), NVP-CGM097, HDM201, MK-8242, RO6839921, DS-3032b, RO5353, RO2468, RO8994, SAR405838, ALRN-6924 (MDM2/MDM4 dual antagonist), one or more MDM2 degraders, or a mixture thereof. Examples of MDM2 degraders include LE-004, LE-102, LE-154, LE-157, LD-222, MD-224, or a combination thereof.

Embodiments of the disclosure include methods of reducing or preventing cancer resistance to one or more BH3 mimetics in an individual, comprising the step of administering to the individual a therapeutically effective amount of one or more agents that target p73 or an isoform thereof. The p73 isoform may be transactivation p73 (Tap73) or ΔNp73, in some cases. In specific embodiments, the method further comprises the step of administering a therapeutically effective amount of one or more BH3 mimetics to the individual. Any method herein may further comprise the step of identifying the level of p73 or an isoform thereof in a sample of cells from the individual.

Particular embodiments include kits comprising any composition encompassed by the disclosure.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

It is specifically contemplated that any limitation discussed with respect to one embodiment of the disclosure may apply to any other embodiment of the disclosure. Furthermore, any composition of the disclosure may be used in any method of the disclosure, and any method of the disclosure may be used to produce or to utilize any composition of the disclosure. Aspects of an embodiment set forth in the Examples are also embodiments that may be implemented in the context of embodiments discussed elsewhere in a different Example or elsewhere in the application, such as in the Brief Summary, Detailed Description, Claims, and Brief Description of the Drawings.

### BRIEF DESCRIPTION OF THE DRAWINGS

For a more complete understanding of the present disclosure, reference is now made to the following descriptions taken in conjunction with the accompanying drawings.

FIGS. 1A-1B. p73 knockdown enhances apoptosis induction by ABT-199 (venetoclax), 5′-azacitidine and combined treatment in a MCL-1 and BCL-2 independent manner.

FIGS. 2A-2B. ABT-199 resistant AML cells show elevated p73 protein levels.

FIGS. 3A-3B. Overexpression of ΔNp73y directly results in ABT-199 resistance.

FIG. 4. Patient-derived xenograft (PDX) AML cells derived from a patient with venetoclax resistance show an elevated p73 protein level.

FIG. 5. Patient-derived (primary) AML samples with venetoclax (VEN) resistance show high p73 protein levels.

FIG. 6. Venetoclax (VEN) resistant primary AML cells show higher ΔNp73 mRNA levels than a VEN sensitive AML sample.

FIG. 7 shows apoptosis induction as measured by annexin V staining (Annexin V positive cells (%)) and live cell numbers as measured by counting beads by flow cytometry. In FIG. 7, prodigiosin (PRD; CAS 82-89-3) is utilized with ABT-199 in ΔNp73 gamma overexpressing MOLM-13 cells.

While various embodiments of the disclosure have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the disclosure described herein may be employed.

### DETAILED DESCRIPTION

### I. Definitions

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.” Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

The term “administration” or “administering,” as used herein, refers to providing, contacting, and/or delivery of an agent by any appropriate route to achieve the desired effect. These agents may be administered to a subject in numerous ways including, but not limited to, orally, ocularly, nasally, intravenously, topically, as aerosols, suppository, etc. and may be used in combination.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “sample” or “test sample” as used herein can mean any sample in which the presence and/or level of an activity, a biomarker, target, agent, or molecule, etc., is to be detected or determined. In some embodiments, the sample is obtained to be analyzed for the presence of p73 or an isoform thereof. In some embodiments, the sample is obtained to be analyzed for the presence of cancer, type of cancer, stage of cancer, and/or metastasis of cancer. Samples may include liquids, solutions, emulsions, mixtures, or suspensions. Samples may include a medical sample. Samples may include any biological fluid or tissue, such as blood, whole blood, fractions of blood such as plasma and serum, peripheral blood mononuclear cells (PBMCs), muscle, interstitial fluid, cheek scrapings, sweat, saliva, urine, tears, synovial fluid, bone marrow, cerebrospinal fluid, nasal secretions, nipple aspirate, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a subject or can be pre-treated, such as by filtration, distillation, extraction, concentration, centrifugation, inactivation of interfering components, addition of reagents, and the like, to modify the character of the sample in some manner as discussed herein or otherwise as is known in the art. Samples may be obtained before treatment, before diagnosis, during treatment, after treatment, or after diagnosis, or a combination thereof. The sample may or may not be stored prior to analysis.

A “therapeutically effective amount,” or “effective dosage,” or “effective amount” as used interchangeably herein unless otherwise defined, means a dosage of an agent or drug effective for periods of time necessary, to achieve the desired therapeutic result. An effective dosage may be determined by a person skilled in the art and may vary according to factors such as the disease state, age, sex, and weight of the individual, and the ability of the drug to elicit a desired response in the individual. This term as used herein may also refer to an amount effective at bringing about a desired in vivo effect in a subject. A therapeutically effective amount may be administered in one or more administrations (e.g., the composition may be given as a preventative treatment or therapeutically at any stage of disease progression, before or after symptoms, and the like), applications, or dosages, and is not intended to be limited to a particular formulation, combination, or administration route. It is within the scope of the present disclosure that the drug may be administered at various times during the course of treatment of the subject. The times of administration and dosages used will depend on several factors, such as the goal of treatment (e.g., treating vs. preventing), condition of the subject, etc. and can be readily determined by one skilled in the art. A therapeutically effective amount is also one in which any toxic or detrimental effects of substance are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The terms “treat,” “treated,” or “treating” as used herein refers to a therapeutic wherein the object is to slow down (lessen) an undesired physiological condition, disorder or disease, or to obtain beneficial or desired clinical results. For the purposes of this disclosure, beneficial or desired clinical results include, but are not limited to, alleviation of one or more symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment also includes prolonging survival as compared to expected survival if not receiving treatment. The terms “treat,” “treated,” or “treating” may include preventing, suppressing, repressing, ameliorating, or completely eliminating the disease. Preventing the disease may involve administering a composition of the present invention to a subject prior to onset of the disease. Suppressing the disease may involve administering a composition of the present invention to a subject after induction of the disease but before its clinical appearance. Repressing or ameliorating the disease may involve administering a composition of the present invention to a subject after clinical appearance of the disease.

The term “BH3 mimetic” as used herein refers to small molecules that mimic the binding to anti-apoptotic BCL-2 family proteins of proteins that comprise the BH3 domain.

The term “isoform” as used herein refers to a member of a set of highly similar proteins that originate from a single gene or gene family and are the result of genetic difference. In specific cases, the isoform is produced as a result of alternative promoter usage and/or differential mRNA splicing. In specific cases, the term “isoform” includes either N- and C-terminal differential proteins (or both) and includes transcript variants having C-terminally differential mRNAs. In certain cases, the isoform may have 1, 2, 3, or more mutations compared to its wild-type counterpart isoform, such as point mutation, deletion, inversion, and so forth.

The term “resistance” as used herein refers to the ability of cancer cells to survive and grow despite being exposed directly or indirectly to one or more BH3 mimetics. The terms “overcoming” or “reducing” with respect to resistance as used herein refers to the ability of at least some cancer cells in the individual to be killed by one or more BH3 mimetics. Such an outcome may be measured by reduction in the number of cancerous cells in blood, blood components, bone marrow, bone marrow components, or in extramedullary tumors for hematologic malignancies, or reduction in tumor load for cancers with solid tumors.

The term “agents that target p73 or an isoform thereof” as used herein refers to agents that reduce or inhibit expression and/or activity of p73 or an isoform thereof. In particular embodiments wherein the agent is targeting a polynucleotide of p73 or an isoform thereof, the agent may or may not directly bind the polynucleotide of p73 or an isoform thereof. In embodiments wherein the agent is targeting a protein of p73 or an isoform thereof, the agent may or may not directly bind the protein of p73 or an isoform thereof.

### II. BH3 Mimetics

Embodiments of the disclosure concern overcoming cancer resistance to one or more anti-apoptotic protein inhibitors, including at least one or more BH3 mimetics. In some cases, a BH3 mimetic is an inhibitor of one or more proteins in the Bcl-2 pathway (including, but not limited to, Bcl-2, Bcl-xL, Mcl-1, Bcl-W, Al/BFL-1, or a combination thereof). The BH3 mimetic may or may not be an inhibitor of Bcl-2. The BH3 mimetic may be a particular combination of inhibitors for the Bcl-2 pathway, including at least a combination of an inhibitor of Bcl-2 and an inhibitor of Bcl-xL, or a combination of an inhibitor of Bcl-2 and an inhibitor of Mcl-1. The BH3 mimetic may be formulated by itself in a pharmaceutically acceptable carrier, or it may be formulated with another active compound, such as being formulated with one or more agents that target p73 or an isoform thereof and/or being formulated with one or more activators of p53.

One or more specific BH3 mimetics may be utilized in cancer treatment and the cancer is resistant to the one or more specific BH3 mimetics. The cancer may be resistant to one or more BH3 mimetics, and the individual may have been treated (and/or is being treated) with one or more of BH3 mimetics that were ineffective because the cancer is resistant to the one or more BH3 mimetics. In some cases, multiple BH3 mimetics were administered to the individual for cancer treatment, and the cancer was resistant to one or more of them.

Examples of BH3 mimetics include AZD5991, ABT-737, ABT-199 (Venetoclax), ABT-263 (Navitoclax), GX15-070 (obatoclax), BI-97CI (sabutoclax), A-1155463, WEHI-539, TW-37, or a mixture thereof. Any one or more of these may be excluded in any embodiment described herein. ABT-263 is also referred to as Navitoclax (4-(4-{[2-(4-Chlorophenyl)-5,5-dimethyl-1-cyclohexen-1-yl]methyl}-1-piperazinyl)-N-[(4-{[(2R)-4-(4-morpholinyl)-1-(phenylsulfanyl)-2-butanyl]amino}-3-[(trifluoromethyl)sulfonyl]phenyl)sulfonyl]benzamide)). ABT-199 is also known as Venetoclax (4-(4-{[2-(4-Chlorophenyl)-4,4-dimethyl-1-cyclohexen-1-yl]methyl}-1-piperazinyl)-N-({[3-nitro-4-[(tetrahydro-2H-pyran-4-ylmethyl)amino]phenyl}sulfonyl)-2-(1H-pyrrolo[2,3-b]pyridin-5-yloxy)benzamide).

Alternative methods of the disclosure include overcoming cancer resistance to inhibitors of Bcl-2, Bcl-xL, Mcl-1, Bcl-W, Al/BFL-1, or a mixture thereof, wherein the inhibitor(s) is not a BH3 mimetic.

III. Agents that Target p73 or an Isoform Thereof

Overcoming cancer resistance to one or more BH3 mimetics is achieved by utilizing one or more agents that target p73 or an isoform thereof. Any composition may utilize one or more agents that target p73 or an isoform thereof, and the agent(s) may be of any type. In specific embodiments, the one or more agents directly target p73 or an isoform thereof. The agent may or may not bind directly to protein that is p73 or an isoform thereof, or the agent may or may not bind directly to a polynucleotide that encodes p73 or an isoform thereof. In specific embodiments, the agent inhibits TAp73 protein activity or inhibits expression of a TAp73-expressing polynucleotide. In some embodiments, the agent inhibits ΔNp73 protein activity or inhibits expression of a ΔNp73-expressing polynucleotide.

A. Targeting of p73 and Related Proteins

In embodiments wherein the protein form of p73 or an isoform thereof is targeted by one or more agents, the protein form may be targeted in any suitable manner. The protein form of p73 or an isoform thereof may be targeted for binding by the agent directly or indirectly. Targeting of the protein form of p73 or an isoform thereof may be in the form of a small molecule that inhibits activity of p73 or an isoform thereof or in the form of an antibody that inhibits activity p73 or an isoform thereof, or it may be a mixture of a small molecule and an antibody.

In cases wherein the agent is an antibody that inhibits activity of p73 or an isoform thereof, the antibody may be of any kind, including monoclonal or polyclonal or a fragment of an antibody, for example. Monoclonal and polyclonal antibodies that target p73 are commercially available, for example.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting. The term “antibody” in specific cases is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference). In specific embodiments, p73 and its isoforms are targeted with single chain antibodies.

Monoclonal antibodies (MAbs) are recognized to have certain advantages, e.g., reproducibility and large-scale production, and their use is generally preferred. The invention thus provides monoclonal antibodies of the human, murine, monkey, rat, hamster, rabbit and even chicken origin. Because of the ease of preparation and ready availability of reagents, murine monoclonal antibodies will often be preferred.

However, “humanized” antibodies are also contemplated, as are chimeric antibodies from mouse, rat, or other species, bearing human constant and/or variable region domains, bispecific antibodies, recombinant and engineered antibodies and fragments thereof. As used herein, the term “humanized” immunoglobulin refers to an immunoglobulin comprising a human framework region and one or more CDR's from a non-human (usually a mouse or rat) immunoglobulin. The non-human immunoglobulin providing the CDR's is called the “donor” and the human immunoglobulin providing the framework is called the “acceptor”. A “humanized antibody” is an antibody comprising a humanized light chain and a humanized heavy chain immunoglobulin.

In embodiments wherein an antibody is the agent that targets p73 or an isoform thereof, the antibody may or may not bind specifically to one variant and not another. For example, an antibody may bind to one particular exon (including any referred to herein) in a variant and not bind to another particular exon in a different variant. The epitope targeted by the antibody may be located in the protein in any region, including the N-terminus, the C-terminus, or there between. In specific embodiments, the epitope for the antibody is located in the transactivation (TA) domain, DNA binding domain (DBD), oligomerization domain (OD), steric alpha motif-like (SAM), or proline-rich (PR) domain(s).

The complexity of the p73 gene produces a variety of isoforms because of alternative promoter usage and differential mRNA splicing and any agent encompassed herein may bind to any isoform encompassed herein. Most alternative splicing occurs at the 3′ end, involving exons 10-13 and generating transcripts encoding protein isoforms with different structures at the C-terminus. The splicing patterns generating C-terminal isoforms p73 α, β, γ, δ, ε, ζ. In addition, there is N-terminal diversity, and such N-terminal isoforms include p73 Δext, p73 Δext/3, ΔNp73, and ΔN′p73, as examples.

B. Targeting of p73 and Related Polynucleotides

In specific embodiments, an agent that targets p73 or an isoform thereof is an agent that targets p73 and related polynucleotides. In some cases, the agent binds to a nucleic acid that encodes the p73 protein or a proteinaceous isoform thereof. In particular cases, the agent binds to an endogenous form of p73 nucleic acid or an isoform nucleic acid thereof, including binds to a mRNA that expresses p73 or a respective isoform. In specific embodiments, a shRNA that hybridizes to p73 or an isoform thereof is an agent utilized in the methods. In specific cases, antisense polynucleotides are utilized. In some cases, the Clustered, Regularly Interspaced, Short Palindromic Repeat (CRISPR) system targets the endogenous nucleic acid encoding p73 or an isoform thereof. In some cases, a small molecule targets p73 and related polynucleotides.

Mechanisms to target p73 and related isoform and variant polynucleotides in specific embodiments utilize shRNA or CRISPR, for example. Such methods utilize knowledge of the sequence of p73 and its isoforms. A particular nucleic acid agent may be selected based on its sequence that is common to multiple p73 isoforms (including some or all isoforms), based on sequence that is common to one or two p73 isoforms, or based on sequence that is unique to one p73 isoform.

In some embodiments, the CRISPR system is utilized in methods of the disclosure to target p73 polynucleotides or isoform polynucleotides polynucleotides. CRISPRs are segments of prokaryotic DNA containing short repetitions of base sequences, followed by short segments of “spacer DNA”. This spacer DNA is foreign DNA obtained from previous exposures to a bacterial virus or plasmid. A set of enzymes called Cas (CRISPR-associated proteins) enzymes are found naturally in association with these CRISPR sequences, and Cas are nucleases that can precisely snip DNA. In nature, bacteria copy the genetic material in each spacer DNA into an RNA molecule. Cas enzymes then take up one of the RNA molecules, which are referred to as the guide RNAs (gRNA). Together they form the CRISPR-Cas system. When the system encounters DNA from a virus that matches the CRISPR RNA, the RNA hybridizes to the DNA sequence and the Cas enzyme then cleaves the DNA in two, preventing the virus from replicating.

There are various Cas enzymes that work in conjunction with CRISPR, but the most well-known and frequently employed in genetic engineering is Cas9 nuclease, which is derived from Streptococcus pyogenes. Together, they form the CRISPR/Cas9 system, called the type II CRISPR system. Cas9 has been shown to be a key player in certain CRISPR mechanisms, specifically type II CRISPR systems where only one Cas protein is required. In this system, the endonuclease Cas9 participates in the cleaving of the target DNA. The Cas9 function is dependent on presence of two nuclease domains, a RuvC-like nuclease domain located at the amino terminus and a HNH-like nuclease domain that resides in the mid-region of the protein.

For site-specific DNA recognition and cleavage, the nuclease Cas9 must complex with two RNA sequences, a crRNA (CRISPR RNA) and a separate trans-activating crRNA (tracrRNA or trRNA), that is partially complementary to the crRNA. The tracrRNA is required for crRNA maturation from a primary transcript encoding multiple pre-crRNAs. This occurs in the presence of RNase III and Cas9. During the cleavage of target DNA, the HNH and RuvC-like nuclease domains of the Cas9 nuclease cut both DNA strands, generating double-stranded breaks (DSBs). The recognition sites are defined by 20-nucleotide target sequence within an associated crRNA transcript. The HNH domain cleaves the complementary strand, while the RuvC domain cleaves the non-complementary strand. The double-stranded endonuclease activity of Cas9 also requires that a short conserved sequence, (2-5 nt) known as Protospacer-Associated Motif (PAM), follows immediately 3′-of the crRNA complementary sequence in the target DNA. The requirement of PAM sequence is obligatory for CRISPR/Cas function.

In general, a two vector system may be used for CRISPR mediated gene editing: 1) a Cas9 endonuclease; and 2) a complex of crRNA (CRISPR RNA) and tracrRNA (trans-activating crRNA). When these two constructs are co-expressed in mammalian cells, they form a complex and are recruited to target DNA sequence. The crRNA and tracrRNA are combined to form a chimeric guide RNA (gRNA) with the same function—to guide Cas9 to target gene sequences.

The adaptation of CRISPR for mammalian cells has revolutionized genome editing with higher accuracy and ease of designing. Unlike ZFN, for example, CRISPR/Cas does not require protein engineering for every gene being targeted. The CRISPR system only requires a few simple DNA constructs to encode the gRNA and Cas9. Although it is rare for a 20 bp gRNA sequence to have 100% homology at multiple sites throughout the genome, sgRNA-Cas9 complexes are tolerant of several mismatches in their targets. Cas9 has been reported to bind multiple locations in genome nonspecifically. However, it creates DNA double strand break only at a handful of those sites. Experimental data also suggests that certain levels of mismatch at the DNA target site allows DNA double strand break. Therefore, strategies for increasing CRISPR/Cas specificity are utilized. One such observation is a point mutation of Aspartate to Alanine (D10A) mutation at the RuvC catalytic domain resulting in single strand breaks (nicks) instead of double strand breaks. The mutant Cas9 is known as Cas9n. Using Cas9n at two neighboring DNA target site allows DNA nicks at close proximity, and if the target sites are appropriately spaced, it creates a double strand break. Therefore, the specificity of DSB creation is higher, which is eventually repaired by NHEJ mechanism. Nonspecifically bound Cas9n creates only nicks which is generally repaired through HR mediated repair and rarely causes mutation or off target effects. In this disclosure, Cas9n and CRISPR are used to knockout Fut8 gene.

In an embodiment of the present disclosure, for methods wherein CRISPR is utilized, the nuclease used in the CRISPR complex is a Cas nuclease, such as Cas9 nuclease or Cas9n nuclease. In an embodiment of the present disclosure, a CRISPR-nuclease construct is a CRISPR Cas construct. In the present disclosure, CRISPR-Cas construct upon expression in a cell provides CRISPR-Cas complex. In the present disclosure, the terms CRISPR-Cas complex and CRISPR-Cas system have the same meaning and scope and are used interchangeably.

In specific embodiments, instead of CRISPR as an agent for targeting p73 or isoform polynucleotides, the methods utilize shRNA for targeting such polynucleotides. The expression “shRNA” refers to a short double-stranded chain wherein a loop is cut into the chain by a dicer and the chain, like siRNA, reacts with RICS so as to express RNAi phenomenon. RNA consists of a stem-loop structure, wherein a long RNA having 19 to 29 nucleotides produces a pair of bases at both sides of the loop site having 5 to 10 nucleotides, thus forming the double-stranded stem. In general, shRNA undergoes in vivo transcription by Pol III promoter and is synthesized, followed by cutting the synthesized shRNA loop using a dicer, and reacting the cut chains with RISC, like siRNA.

According to one embodiment of the present disclosure, in order to reduce p73 (or isoform) expression, a vector encoding the anti-sense base sequence is provided. Such a system enables more stable and continuous expression of siRNA for a long period of time. A process of preparing shRNA and introducing the same into a cell or an animal may depend on cell-biological performances of target gene products and/or purposes of studies, and all of siRNAs or shRNAs in association with p73 (or isoform) gene may inhibit expression of p73 and related proteins.

Furthermore, the present disclosure may provide a method of overcoming resistance to cancer in mammalian cells such as human cells or in vitro established mammalian cell-lines, comprising administering p73 (or isoform)-targeting shRNA into the cells, as well as a method for prevention or treatment of cancer resistance comprising administering the foregoing shRNA to cells. In one embodiment, the administering process of the shRNA to the cell is to introduce a vector comprising the shRNA sequence into the cell and may include any conventional methods used by persons skilled in the art. The foregoing cell may be a mammalian cell such as the human cell.

Thus, in some cases the polynucleotide of any p73 or isoform thereof may be targeted with one or more agents that specifically target the polynucleotide based on a particular sequence of the targeted polynucleotide. Polynucleotides that encode p73 or isoform thereof are known in the art, but examples are provided below, as are examples of respective encoded proteins.

In some cases, the agent is a nucleic acid that targets variants arisen from diversity at the N-terminus. As one example, the ΔNp73 has different variants (such as different transcript variants), and examples of sequences that may be targeted include at least the following (and including the respective GenBank® Accession numbers):

ΔNp73 alpha
NM_001126240 Homo sapiens tumor protein p73 (TP73), transcript variant 2, mRNA.
DeltaNp73 alpha
CDS 235..1998
atgctgtacgtcggtgaccccgcacggcacctcgccacggcccagttcaatctgctgagcagcaccatggaccagat
gagcagccgcgcggcctcggccagcccctacaccccagagcacgccgccagcgtgcccacccactcgccctacgcac
aacccagctccaccttcgacaccatgtcgccggcgcctgtcatcccctccaacaccgactaccccggaccccaccac
tttgaggtcactttccagcagtccagcacggccaagtcagccacctggacgtactccccgctcttgaagaaactcta
ctgccagatcgccaagacatgccccatccagatcaaggtgtccaccccgccacccccaggcaccgccatccgggcca
tgcctgtttacaagaaagcggagcacgtgaccgacgtcgtgaaacgctgccccaaccacgagctcgggagggacttc
aacgaaggacagtctgctccagccagccacctcatccgcgtggaaggcaataatctctcgcagtatgtggatgaccc
tgtcaccggcaggcagagcgtcgtggtgccctatgagccaccacaggtggggacggaattcaccaccatcctgtaca
acttcatgtgtaacagcagctgtgtagggggcatgaaccggcggcccatcctcatcatcatcaccctggagatgcgg
gatgggcaggtgctgggccgccggtcctttgagggccgcatctgcgcctgtcctggccgcgaccgaaaagctgatga
ggaccactaccgggagcagcaggccctgaacgagagctccgccaagaacggggccgccagcaagcgtgccttcaagc
agagcccccctgccgtccccgcccttggtgccggtgtgaagaagcggcggcatggagacgaggacacgtactacctt
caggtgcgaggccgggagaactttgagatcctgatgaagctgaaagagagcctggagctgatggagttggtgccgca
gccactggtggactcctatcggcagcagcagcagctcctacagaggccgagtcacctacagcccccgtcctacgggc
cggtcctctcgcccatgaacaaggtgcacgggggcatgaacaagctgccctccgtcaaccagctggtgggccagcct
cccccgcacagttcggcagctacacccaacctggggcccgtgggccccgggatgctcaacaaccatggccacgcagt
gccagccaacggcgagatgagcagcagccacagcgcccagtccatggtctcggggtcccactgcactccgccacccc
cctaccacgccgaccccagcctcgtcagttttttaacaggattggggtgtccaaactgcatcgagtatttcacctcc
caagggttacagagcatttaccacctgcagaacctgaccattgaggacctgggggccctgaagatccccgagcagta
ccgcatgaccatctggcggggcctgcaggacctgaagcagggccacgactacagcaccgcgcagcagctgctccgct
ctagcaacgcggccaccatctccatcggcggctcaggggaactgcagcgccagcgggtcatggaggccgtgcacttc
cgcgtgcgccacaccatcaccatccccaaccgcggcggcccaggcggcggccctgacgagtgggcggacttcggctt
cgacctgcccgactgcaaggcccgcaagcagcccatcaaggaggagttcacggaggccgagatccactga (SEQ
ID NO: 1)
Amino acid sequence
NP_001119712 587 aa

| | |
|---|---|
| 1 | mlyvgdparhlataqfnllsstmdqmssraasaspytpehaasvpthspyaqpsstfdtm |
| 61 | spapvipsntdypgphhfevtfqqsstaksatwtyspllkklycqiaktcpiqikvstpp |
| 121 | ppgtairampvykkaehvtdvvkrcpnhelgrdfnegqsapashlirvegnnlsqyvddp |
| 181 | vtgrqsvvvpyeppqvgtefttilynfmcnsscvggmnrrpiliiitlemrdgqvlgrrs |
| 241 | fegricacpgrdrkadedhyreqqalnessakngaaskrafkqsppavpalgagvkkrrh |
| 301 | gdedtyylqvrgrenfeilmklkeslelmelvpqplvdsyrqqqqllqrpshlqppsygp |
| 361 | vlspmnkvhggmnklpsvnqlvgqppphssaatpnlgpvgpgmlnnhghavpangemsss |
| 421 | hsaqsmvsgshctppppyhadpslvsfltglgcpncieyftsqglqsiyhlqnltiedlg |
| 481 | alkipeqyrmtiwrglqdlkqghdystaqqllrssnaatisiggsgelqrqrvmeavhfr |
| 541 | vrhtitipnrggpgggpdewadfgfdlpdckarkqpikeefteaeih (SEQ ID NO: 2) |

ΔNp73 beta
NM_001126241 Homo sapiens tumor protein p73 (TP73), transcript variant 3, mRNA.
DNp73 beta, CDS 235..1587
atgctgtacgtcggtgaccccgcacggcacctcgccacggcccagttcaatctgctgagcagcaccatggaccagat
gagcagccgcgcggcctcggccagcccctacaccccagagcacgccgccagcgtgcccacccactcgccctacgcac
aacccagctccaccttcgacaccatgtcgccggcgcctgtcatcccctccaacaccgactaccccggaccccaccac
tttgaggtcactttccagcagtccagcacggccaagtcagccacctggacgtactccccgctcttgaagaaactcta
ctgccagatcgccaagacatgccccatccagatcaaggtgtccaccccgccacccccaggcaccgccatccgggcca
tgcctgtttacaagaaagcggagcacgtgaccgacgtcgtgaaacgctgccccaaccacgagctcgggagggacttc
aacgaaggacagtctgctccagccagccacctcatccgcgtggaaggcaataatctctcgcagtatgtggatgaccc
tgtcaccggcaggcagagcgtcgtggtgccctatgagccaccacaggtggggacggaattcaccaccatcctgtaca
acttcatgtgtaacagcagctgtgtagggggcatgaaccggcggcccatcctcatcatcatcaccctggagatgcgg
gatgggcaggtgctgggccgccggtcctttgagggccgcatctgcgcctgtcctggccgcgaccgaaaagctgatga
ggaccactaccgggagcagcaggccctgaacgagagctccgccaagaacggggccgccagcaagcgtgccttcaagc
agagcccccctgccgtccccgcccttggtgccggtgtgaagaagcggcggcatggagacgaggacacgtactacctt
caggtgcgaggccgggagaactttgagatcctgatgaagctgaaagagagcctggagctgatggagttggtgccgca
gccactggtggactcctatcggcagcagcagcagctcctacagaggccgagtcacctacagcccccgtcctacgggc
cggtcctctcgcccatgaacaaggtgcacgggggcatgaacaagctgccctccgtcaaccagctggtgggccagcct
cccccgcacagttcggcagctacacccaacctggggcccgtgggccccgggatgctcaacaaccatggccacgcagt
gccagccaacggcgagatgagcagcagccacagcgcccagtccatggtctcggggtcccactgcactccgccacccc
cctaccacgccgaccccagcctcgtcaggacctgggggccctga (SEQ ID NO: 3)
Amino acid sequence
NP_001119713 450 aa

| | |
|---|---|
| 1 | mlyvgdparh lataqfnlls stmdqmssra asaspytpeh aasvpthspy aqpsstfdtm |
| 61 | spapvipsnt dypgphhfev tfqqsstaks atwtyspllk klycqiaktc piqikvstpp |
| 121 | ppgtairamp vykkaehvtd vvkrcpnhel grdfnegqsa pashlirveg nnlsqyvddp |
| 181 | vtgrqsvvvp yeppqvgtef ttilynfmcn sscvggmnrr piliiitlem rdgqvlgrrs |
| 241 | fegricacpg rdrkadedhy reqqalness akngaaskra fkqsppavpa igagvkkrrh |
| 301 | gdedtyylqv rgrenfeilm klkeslelme ivpqplvdsy rqqqqllqrp shlqppsygp |
| 361 | vlspmnkvhg gmnklpsvnq ivgqppphss aatpnlgpvg pgminnhgha vpangemsss |
| 421 | hsaqsmvsgs hctppppyha dpslvrtwgp (SEQ ID NO: 4) |

ΔNp73 gamma
NM_001126242 Homo sapiens tumor protein p73 (TP73), transcript variant 4,
DeltaNp73 gamma
CDS 235..1515
atgctgtacgtcggtgaccccgcacggcacctcgccacggcccagttcaatctgctgagcagcaccatggaccagat
gagcagccgcgcggcctcggccagcccctacaccccagagcacgccgccagcgtgcccacccactcgccctacgcac
aacccagctccaccttcgacaccatgtcgccggcgcctgtcatcccctccaacaccgactaccccggaccccaccac
tttgaggtcactttccagcagtccagcacggccaagtcagccacctggacgtactccccgctcttgaagaaactcta
ctgccagatcgccaagacatgccccatccagatcaaggtgtccaccccgccacccccaggcaccgccatccgggcca
tgcctgtttacaagaaagcggagcacgtgaccgacgtcgtgaaacgctgccccaaccacgagctcgggagggacttc
aacgaaggacagtctgctccagccagccacctcatccgcgtggaaggcaataatctctcgcagtatgtggatgaccc
tgtcaccggcaggcagagcgtcgtggtgccctatgagccaccacaggtggggacggaattcaccaccatcctgtaca
acttcatgtgtaacagcagctgtgtagggggcatgaaccggcggcccatcctcatcatcatcaccctggagatgcgg
gatgggcaggtgctgggccgccggtcctttgagggccgcatctgcgcctgtcctggccgcgaccgaaaagctgatga
ggaccactaccgggagcagcaggccctgaacgagagctccgccaagaacggggccgccagcaagcgtgccttcaagc
agagcccccctgccgtccccgcccttggtgccggtgtgaagaagcggcggcatggagacgaggacacgtactacctt
caggtgcgaggccgggagaactttgagatcctgatgaagctgaaagagagcctggagctgatggagttggtgccgca
gccactggtggactcctatcggcagcagcagcagctcctacagaggccgccccgggatgctcaacaaccatggccac
gcagtgccagccaacggcgagatgagcagcagccacagcgcccagtccatggtctcggggtcccactgcactccgcc
acccccctaccacgccgaccccagcctcgtcagttttttaacaggattggggtgtccaaactgcatcgagtatttca
cctcccaagggttacagagcatttaccacctgcagaacctgaccattga (SEQ ID NO: 5)
Amino acid sequence
NP_001119714 426 aa

| | |
|---|---|
| 1 | mlyvgdparh lataqfnlls stmdqmssra asaspytpeh aasvpthspy aqpsstfdtm |
| 61 | spapvipsnt dypgphhfev tfqqsstaks atwtyspllk klycqiaktc piqikvstpp |
| 121 | ppgtairamp vykkaehvtd vvkrcpnhel grdfnegqsa pashlirveg nnlsqyvddp |
| 181 | vtgrqsvvvp yeppqvgtef ttilynfmcn sscvggmnrr piliiitlem rdgqvlgrrs |
| 241 | fegricacpg rdrkadedhy reqqalness akngaaskra fkqsppavpa igagvkkrrh |
| 301 | gdedtyylqv rgrenfeilm klkeslelme ivpqplvdsy rqqqqllqrp prdaqqpwpr |
| 361 | sasqrrdeqq pqrpvhglgv plhsatplpr rpqprqffnr igvsklhrvf hlprvtehlp |
| 421 | paepdh (SEQ ID NO: 6) |

ΔNp73 delta
NM_001204189 Homo sapiens tumor protein p73 (TP73), transcript variant 5, mRNA.
DeltaNp73 delta
CDS 235..1299
Atgctgtacgtcggtgaccccgcacggcacctcgccacggcccagttcaatctgctgagcagcaccatggaccagat
gagcagccgcgcggcctcggccagcccctacaccccagagcacgccgccagcgtgcccacccactcgccctacgcac
aacccagctccaccttcgacaccatgtcgccggcgcctgtcatcccctccaacaccgactaccccggaccccaccac
tttgaggtcactttccagcagtccagcacggccaagtcagccacctggacgtactccccgctcttgaagaaactcta
ctgccagatcgccaagacatgccccatccagatcaaggtgtccaccccgccacccccaggcaccgccatccgggcca
tgcctgtttacaagaaagcggagcacgtgaccgacgtcgtgaaacgctgccccaaccacgagctcgggagggacttc
aacgaaggacagtctgctccagccagccacctcatccgcgtggaaggcaataatctctcgcagtatgtggatgaccc
tgtcaccggcaggcagagcgtcgtggtgccctatgagccaccacaggtggggacggaattcaccaccatcctgtaca
acttcatgtgtaacagcagctgtgtagggggcatgaaccggcggcccatcctcatcatcatcaccctggagatgcgg
gatgggcaggtgctgggccgccggtcctttgagggccgcatctgcgcctgtcctggccgcgaccgaaaagctgatga
ggaccactaccgggagcagcaggccctgaacgagagctccgccaagaacggggccgccagcaagcgtgccttcaagc
agagcccccctgccgtccccgcccttggtgccggtgtgaagaagcggcggcatggagacgaggacacgtactacctt
caggtgcgaggccgggagaactttgagatcctgatgaagctgaaagagagcctggagctgatggagttggtgccgca
gccactggtggactcctatcggcagcagcagcagctcctacagaggccgacctgggggccctga (SEQ ID NO: 7)
Amino acid sequence
NP_001191118 354 aa

| | |
|---|---|
| 1 | mlyvgdparh lataqfnlls stmdqmssra asaspytpeh aasvpthspy aqpsstfdtm |
| 61 | spapvipsnt dypgphhfev tfqqsstaks atwtyspllk klycqiaktc piqikvstpp |
| 121 | ppgtairamp vykkaehvtd vvkrcpnhel grdfnegqsa pashlirveg nnlsqyvddp |
| 181 | vtgrqsvvvp yeppqvgtef ttilynfmcn sscvggmnrr pililitlem rdgqvlgrrs |
| 241 | fegricacpg rdrkadedhy reqqalness akngaaskra fkqsppavpa lgagvkkrrh |
| 301 | gdedtyylqv rgrenfeilm klkeslelme ivpqplvdsy rqqqqllqrp twgp (SEQ ID NO: 8) |

ΔNp73 epsilon
NM_001204190 Homo sapiens tumor protein p73 (TP73), transcript variant 6, mRNA.
DeltaNp73 epsilon CDS 235..1755
atgctgtacgtcggtgaccccgcacggcacctcgccacggcccagttcaatctgctgagcagcaccatggaccagat
gagcagccgcgcggcctcggccagcccctacaccccagagcacgccgccagcgtgcccacccactcgccctacgcac
aacccagctccaccttcgacaccatgtcgccggcgcctgtcatcccctccaacaccgactaccccggaccccaccac
tttgaggtcactttccagcagtccagcacggccaagtcagccacctggacgtactccccgctcttgaagaaactcta
ctgccagatcgccaagacatgccccatccagatcaaggtgtccaccccgccacccccaggcaccgccatccgggcca
tgcctgtttacaagaaagcggagcacgtgaccgacgtcgtgaaacgctgccccaaccacgagctcgggagggacttc
aacgaaggacagtctgctccagccagccacctcatccgcgtggaaggcaataatctctcgcagtatgtggatgaccc
tgtcaccggcaggcagagcgtcgtggtgccctatgagccaccacaggtggggacggaattcaccaccatcctgtaca
acttcatgtgtaacagcagctgtgtagggggcatgaaccggcggcccatcctcatcatcatcaccctggagatgcgg
gatgggcaggtgctgggccgccggtcctttgagggccgcatctgcgcctgtcctggccgcgaccgaaaagctgatga
ggaccactaccgggagcagcaggccctgaacgagagctccgccaagaacggggccgccagcaagcgtgccttcaagc
agagcccccctgccgtccccgcccttggtgccggtgtgaagaagcggcggcatggagacgaggacacgtactacctt
caggtgcgaggccgggagaactttgagatcctgatgaagctgaaagagagcctggagctgatggagttggtgccgca
gccactggtggactcctatcggcagcagcagcagctcctacagaggccgccccgggatgctcaacaaccatggccac
gcagtgccagccaacggcgagatgagcagcagccacagcgcccagtccatggtctcggggtcccactgcactccgcc
acccccctaccacgccgaccccagcctcgtcaggacctgggggccctgaagatccccgagcagtaccgcatgaccat
ctggcggggcctgcaggacctgaagcagggccacgactacagcaccgcgcagcagctgctccgctctagcaacgcgg
ccaccatctccatcggcggctcaggggaactgcagcgccagcgggtcatggaggccgtgcacttccgcgtgcgccac
accatcaccatccccaaccgcggcggcccaggcggcggccctgacgagtgggcggacttcggcttcgacctgcccga
ctgcaaggcccgcaagcagcccatcaaggaggagttcacggaggccgagatccactga (SEQ ID NO: 9)
Amino acid sequence
NP_001191119 506 aa

| | |
|---|---|
| 1 | mlyvgdparh lataqfnlls stmdqmssra asaspytpeh aasvpthspy aqpsstfdtm |
| 61 | spapvipsnt dypgphhfev tfqqsstaks atwtyspllk klycqiaktc piqikvstpp |
| 121 | ppgtairamp vykkaehvtd vvkrcpnhel grdfnegqsa pashlirveg nnlsqyvddp |
| 181 | vtgrqsvvvp yeppqvgtef ttilynfmcn sscvggmnrr pililitlem rdgqvlgrrs |
| 241 | fegricacpg rdrkadedhy reqqalness akngaaskra fkqsppavpa lgagvkkrrh |
| 301 | gdedtyylqv rgrenfeilm klkeslelme lvpqplvdsy rqqqqllqrp prdaqqpwpr |
| 361 | sasqrrdeqq pqrpvhqlqv plhsatplpr rpqprqdlqa lkipeqyrmt iwrqlqdlkq |
| 421 | ghdystaqql irssnaatis iggsgelqrq rvmeavhfrv rhtitipnrg gpgggpdewa |
| 481 | dfgfdlpdck arkqpikeef teaeih (SEQ ID NO: 10) |

ΔNp73 zeta
NM_001204191 Homo sapiens tumor protein p73 (TP73), transcript variant 7, mRNA.
DeltaNp73 zeta CDS 235..1710
atgctgtacgtcggtgaccccgcacggcacctcgccacggcccagttcaatctgctgagcagcaccatggaccagat
gagcagccgcgcggcctcggccagcccctacaccccagagcacgccgccagcgtgcccacccactcgccctacgcac
aacccagctccaccttcgacaccatgtcgccggcgcctgtcatcccctccaacaccgactaccccggaccccaccac
tttgaggtcactttccagcagtccagcacggccaagtcagccacctggacgtactccccgctcttgaagaaactcta
ctgccagatcgccaagacatgccccatccagatcaaggtgtccaccccgccacccccaggcaccgccatccgggcca
tgcctgtttacaagaaagcggagcacgtgaccgacgtcgtgaaacgctgccccaaccacgagctcgggagggacttc
aacgaaggacagtctgctccagccagccacctcatccgcgtggaaggcaataatctctcgcagtatgtggatgaccc
tgtcaccggcaggcagagcgtcgtggtgccctatgagccaccacaggtggggacggaattcaccaccatcctgtaca
acttcatgtgtaacagcagctgtgtagggggcatgaaccggcggcccatcctcatcatcatcaccctggagatgcgg
gatgggcaggtgctgggccgccggtcctttgagggccgcatctgcgcctgtcctggccgcgaccgaaaagctgatga
ggaccactaccgggagcagcaggccctgaacgagagctccgccaagaacggggccgccagcaagcgtgccttcaagc
agagcccccctgccgtccccgcccttggtgccggtgtgaagaagcggcggcatggagacgaggacacgtactacctt
caggtgcgaggccgggagaactttgagatcctgatgaagctgaaagagagcctggagctgatggagttggtgccgca
gccactggtggactcctatcggcagcagcagcagctcctacagaggccttttttaacaggattggggtgtccaaact
gcatcgagtatttcacctcccaagggttacagagcatttaccacctgcagaacctgaccattgaggacctgggggcc
ctgaagatccccgagcagtaccgcatgaccatctggcggggcctgcaggacctgaagcagggccacgactacagcac
cgcgcagcagctgctccgctctagcaacgcggccaccatctccatcggcggctcaggggaactgcagcgccagcggg
tcatggaggccgtgcacttccgcgtgcgccacaccatcaccatccccaaccgcggcggcccaggcggcggccctgac
gagtgggcggacttcggcttcgacctgcccgactgcaaggcccgcaagcagcccatcaaggaggagttcacggaggc
cgagatccactga (SEQ ID NO: 11)
Amino acid sequence
NP_001191120 491 aa

| | |
|---|---|
| 1 | mlyvgdparh lataqfnlls stmdqmssra asaspytpeh aasvpthspy aqpsstfdtm |
| 61 | spapvipsnt dypgphhfev tfqqsstaks atwtyspllk klycqiaktc piqikvstpp |
| 121 | ppgtairamp vykkaehvtd vvkrcpnhel grdfnegqsa pashlirveg nnlsqyvddp |
| 181 | vtgrqsvvvp yeppqvgtef ttilynfmcn sscvggmnrr piliiitlem rdgqvlgrrs |
| 241 | fegricacpg rdrkadedhy reqqalness akngaaskra fkqsppavpa igagvkkrrh |
| 301 | gdedtyylqv rgrenfeilm klkeslelme ivpqplvdsy rqqqqllqrp fltglgcpnc |
| 361 | ieyftsqglq siyhlqnlti edlgalkipe qyrmtiwrgl qdlkqghdys taqqllrssn |
| 421 | aatisiggsg elqrqrvmea vhfrvrhtit ipnrggpggg pdewadfgfd lpdckarkqp |
| 481 | ikeefteaei h (SEQ ID NO: 12) |

In some cases, the agent is a nucleic acid that targets variants arisen from diversity at the C-terminus. As one example, the TAp73 has different variants (such as different transcript variants), and examples of sequences that may be targeted include at least the following (and including the respective GenBank® Accession numbers):

TAp73 alpha
NM_005427 5154 bp
Homo sapiens tumor protein p73 (TP73), transcript variant 1
TAp73 alpha
CDS 111..2021
atggcccagtccaccgccacctcccctgatgggggcaccacgtttgagcacctctggagctctctggaaccagacag
cacctacttcgaccttccccagtcaagccgggggaataatgaggtggtgggcggaacggattccagcatggacgtct
tccacctggagggcatgactacatctgtcatggcccagttcaatctgctgagcagcaccatggaccagatgagcagc
cgcgcggcctcggccagcccctacaccccagagcacgccgccagcgtgcccacccactcgccctacgcacaacccag
ctccaccttcgacaccatgtcgccggcgcctgtcatcccctccaacaccgactaccccggaccccaccactttgagg
tcactttccagcagtccagcacggccaagtcagccacctggacgtactccccgctcttgaagaaactctactgccag
atcgccaagacatgccccatccagatcaaggtgtccaccccgccacccccaggcaccgccatccgggccatgcctgt
ttacaagaaagcggagcacgtgaccgacgtcgtgaaacgctgccccaaccacgagctcgggagggacttcaacgaag
gacagtctgctccagccagccacctcatccgcgtggaaggcaataatctctcgcagtatgtggatgaccctgtcacc
ggcaggcagagcgtcgtggtgccctatgagccaccacaggtggggacggaattcaccaccatcctgtacaacttcat
gtgtaacagcagctgtgtagggggcatgaaccggcggcccatcctcatcatcatcaccctggagatgcgggatgggc
aggtgctgggccgccggtcctttgagggccgcatctgcgcctgtcctggccgcgaccgaaaagctgatgaggaccac
taccgggagcagcaggccctgaacgagagctccgccaagaacggggccgccagcaagcgtgccttcaagcagagccc
ccctgccgtccccgcccttggtgccggtgtgaagaagcggcggcatggagacgaggacacgtactaccttcaggtgc
gaggccgggagaactttgagatcctgatgaagctgaaagagagcctggagctgatggagttggtgccgcagccactg
gtggactcctatcggcagcagcagcagctcctacagaggccgagtcacctacagcccccgtcctacgggccggtcct
ctcgcccatgaacaaggtgcacgggggcatgaacaagctgccctccgtcaaccagctggtgggccagcctcccccgc
acagttcggcagctacacccaacctggggcccgtgggccccgggatgctcaacaaccatggccacgcagtgccagcc
aacggcgagatgagcagcagccacagcgcccagtccatggtctcggggtcccactgcactccgccacccccctacca
cgccgaccccagcctcgtcagttttttaacaggattggggtgtccaaactgcatcgagtatttcacctcccaagggt
tacagagcatttaccacctgcagaacctgaccattgaggacctgggggccctgaagatccccgagcagtaccgcatg
accatctggcggggcctgcaggacctgaagcagggccacgactacagcaccgcgcagcagctgctccgctctagcaa
cgcggccaccatctccatcggcggctcaggggaactgcagcgccagcgggtcatggaggccgtgcacttccgcgtgc
gccacaccatcaccatccccaaccgcggcggcccaggcggcggccctgacgagtgggcggacttcggcttcgacctg
cccgactgcaaggcccgcaagcagcccatcaaggaggagttcacggaggccgagatccactga (SEQ ID NO: 13)
LOCUS NP_005418 636 aa

| | |
|---|---|
| 1 | maqstatspd ggttfehlws slepdstyfd ipqssrgnne vvggtdssmd vfhlegmtts |
| 61 | vmaqfnllss tmdqmssraa saspytpeha asvpthspya qpsstfdtms papvipsntd |
| 121 | ypgphhfevt fqqsstaksa twtysplikk lycqiaktcp iqikvstppp pgtairampv |
| 181 | ykkaehvtdv vkrcpnhelg rdfnegqsap ashlirvegn nlsqyvddpv tgrqsvvvpy |
| 241 | eppqvgteft tilynfmcns scvggmnrrp iliiitlemr dgqvlgrrsf egricacpgr |
| 301 | drkadedhyr eqqalnessa kngaaskraf kqsppavpal gagvkkrrhg dedtyylqvr |
| 361 | grenfeilmk ikeslelmel vpqplvdsyr qqqqllqrps hlqppsygpv ispmnkvhgg |
| 421 | mnklpsvnql vgqppphssa atpnlgpvgp gminnhghav pangemsssh saqsmvsgsh |
| 481 | ctppppyhad pslvsfltgl gcpncieyft sqglqsiyhl qnltiedlga lkipeqyrmt |
| 541 | iwrglqdlkq ghdystaqql lrssnaatis iggsgelqrq rvmeavhfrv rhtitipnrg |
| 601 | gpgggpdewa dfgfdlpdck arkgpikeef teaeih (SEQ ID NO: 14) |

TAp73 beta
TAp73 beta CDS
LOCUS NM_001204184 5098 bp mRNA linear PRI 07-AUG-2019
DEFINITION Homo sapiens tumor protein p73 (TP73), transcript variant 8, mRNA.
ACCESSION NM_001204184
VERSION NM_001204184.2
atggcccagtccaccgccacctcccctgatgggggcaccacgtttgagcacctctggagctctctggaaccagacag
cacctacttcgaccttccccagtcaagccgggggaataatgaggtggtgggcggaacggattccagcatggacgtct
tccacctggagggcatgactacatctgtcatggcccagttcaatctgctgagcagcaccatggaccagatgagcagc
cgcgcggcctcggccagcccctacaccccagagcacgccgccagcgtgcccacccactcgccctacgcacaacccag
ctccaccttcgacaccatgtcgccggcgcctgtcatcccctccaacaccgactaccccggaccccaccactttgagg
tcactttccagcagtccagcacggccaagtcagccacctggacgtactccccgctcttgaagaaactctactgccag
atcgccaagacatgccccatccagatcaaggtgtccaccccgccacccccaggcaccgccatccgggccatgcctgt
ttacaagaaagcggagcacgtgaccgacgtcgtgaaacgctgccccaaccacgagctcgggagggacttcaacgaag
gacagtctgctccagccagccacctcatccgcgtggaaggcaataatctctcgcagtatgtggatgaccctgtcacc
ggcaggcagagcgtcgtggtgccctatgagccaccacaggtggggacggaattcaccaccatcctgtacaacttcat
gtgtaacagcagctgtgtagggggcatgaaccggcggcccatcctcatcatcatcaccctggagatgcgggatgggc
aggtgctgggccgccggtcctttgagggccgcatctgcgcctgtcctggccgcgaccgaaaagctgatgaggaccac
taccgggagcagcaggccctgaacgagagctccgccaagaacggggccgccagcaagcgtgccttcaagcagagccc
ccctgccgtccccgcccttggtgccggtgtgaagaagcggcggcatggagacgaggacacgtactaccttcaggtgc
gaggccgggagaactttgagatcctgatgaagctgaaagagagcctggagctgatggagttggtgccgcagccactg
gtggactcctatcggcagcagcagcagctcctacagaggccgagtcacctacagcccccgtcctacgggccggtcct
ctcgcccatgaacaaggtgcacgggggcatgaacaagctgccctccgtcaaccagctggtgggccagcctcccccgc
acagttcggcagctacacccaacctggggcccgtgggccccgggatgctcaacaaccatggccacgcagtgccagcc
aacggcgagatgagcagcagccacagcgcccagtccatggtctcggggtcccactgcactccgccacccccctacca
cgccgaccccagcctcgtcaggacctgggggccctga (SEQ ID NO: 15)
Amino acid sequence (499 amino acids) NP_001191113.1
MAQSTATSPDGGTTFEHLWSSLEPDSTYFDLPQSSRGNNEWGGTDSSMDVFHLEGMTTSVMAQFNLLSST
MDQMSSRAASASPYTPEHAASVPTHSPYAQPSSTFDTMSPAPVIPSNTDYPGPHHFEVTFQQSSTAKSATWTYSPLL
KKLYCQIAKTCPIQIKVSTPPPPGTAIRAMPVYKKAEHVTDVVKRCPNHELGRDFNEGQSAPASHLIRVEGNNLSQY
VDDPVTGRQSVVVPYEPPQVGTEFTTILYNFMCNSSCVGGMNRRPILIIITLEMRDGQVLGRRSFEGRICACPGRDR
KADEDHYREQQALNESSAKNGAASKRAFKQSPPAVPALGAGVKKRRHGDEDTYYLQVRGRENFEILMKLKESLELME
LVPQPLVDSYRQQQQLLQRPSHLQPPSYGPVLSPMNKVHGGMNKLPSVNQLVGQPPPHSSAATPNLGPVGPGMLNNH
GHAVPANGEMSSSHSAQSMVSGSHCTPPPPYHADPSLVRTWGP (SEQ ID NO: 16)
TAp73 gamma
TAp73 gamma CDS.
LOCUS NM_001204185 5043 bp mRNA linear PRI 07-AUG-2019
DEFINITION Homo sapiens tumor protein p73 (TP73), transcript variant 9, mRNA.
ACCESSION NM 001204185
VERSION NM_001204185.2
atggcccagtccaccgccacctcccctgatgggggcaccacgtttgagcacctctggagctctctggaaccagacag
cacctacttcgaccttccccagtcaagccgggggaataatgaggtggtgggcggaacggattccagcatggacgtct
tccacctggagggcatgactacatctgtcatggcccagttcaatctgctgagcagcaccatggaccagatgagcagc
cgcgcggcctcggccagcccctacaccccagagcacgccgccagcgtgcccacccactcgccctacgcacaacccag
ctccaccttcgacaccatgtcgccggcgcctgtcatcccctccaacaccgactaccccggaccccaccactttgagg
tcactttccagcagtccagcacggccaagtcagccacctggacgtactccccgctcttgaagaaactctactgccag
atcgccaagacatgccccatccagatcaaggtgtccaccccgccacccccaggcaccgccatccgggccatgcctgt
ttacaagaaagcggagcacgtgaccgacgtcgtgaaacgctgccccaaccacgagctcgggagggacttcaacgaag
gacagtctgctccagccagccacctcatccgcgtggaaggcaataatctctcgcagtatgtggatgaccctgtcacc
ggcaggcagagcgtcgtggtgccctatgagccaccacaggtggggacggaattcaccaccatcctgtacaacttcat
gtgtaacagcagctgtgtagggggcatgaaccggcggcccatcctcatcatcatcaccctggagatgcgggatgggc
aggtgctgggccgccggtcctttgagggccgcatctgcgcctgtcctggccgcgaccgaaaagctgatgaggaccac
taccgggagcagcaggccctgaacgagagctccgccaagaacggggccgccagcaagcgtgccttcaagcagagccc
ccctgccgtccccgcccttggtgccggtgtgaagaagcggcggcatggagacgaggacacgtactaccttcaggtgc
gaggccgggagaactttgagatcctgatgaagctgaaagagagcctggagctgatggagttggtgccgcagccactg
gtggactcctatcggcagcagcagcagctcctacagaggccgccccgggatgctcaacaaccatggccacgcagtgc
cagccaacggcgagatgagcagcagccacagcgcccagtccatggtctcggggtcccactgcactccgccacccccc
taccacgccgaccccagcctcgtcagttttttaacaggattggggtgtccaaactgcatcgagtatttcacctccca
agggttacagagcatttaccacctgcagaacctgaccattga (SEQ ID NO: 17)
Amino acid sequence (476 amino acids) NP_001191114.1
MAQSTATSPDGGTTFEHLWSSLEPDSTYFDLPQSSRGNNEWGGTDSSMDVFHLEGMTTSVMAQFNLLSST
MDQMSSRAASASPYTPEHAASVPTHSPYAQPSSTFDTMSPAPVIPSNTDYPGPHHFEVTFQQSSTAKSATWTYSPLL
KKLYCQIAKTCPIQIKVSTPPPPGTAIRAMPVYKKAEHVTDVVKRCPNHELGRDFNEGQSAPASHLIRVEGNNLSQY
VDDPVTGRQSVVVPYEPPQVGTEFTTILYNFMCNSSCVGGMNRRPILIIITLEMRDGQVLGRRSFEGRICACPGRDR
KADEDHYREQQALNESSAKNGAASKRAFKQSPPAVPALGAGVKKRRHGDEDTYYLQVRGRENFEILMKLKESLELME
LVPQPLVDSYRQQQQLLQRPPRDAQQPWPRSASQQRRDEQQPQRPVHGLGVPLHSATPLPRRPQPRQFFNRIGVSKL
HRVFHLPRVTEHLPPAEPDH (SEQ ID NO: 18)
TAp73 delta
TAp73 delta CDS.
LOCUS NM_001204186 4810 bp mRNA linear PRI 08-AUG-2019
DEFINITION Homo sapiens tumor protein p73 (TP73), transcript variant 10, mRNA.
ACCESSION NM_001204186
VERSION NM_001204186.2
atggcccagtccaccgccacctcccctgatgggggcaccacgtttgagcacctctggagctctctggaaccagacag
cacctacttcgaccttccccagtcaagccgggggaataatgaggtggtgggcggaacggattccagcatggacgtct
tccacctggagggcatgactacatctgtcatggcccagttcaatctgctgagcagcaccatggaccagatgagcagc
cgcgcggcctcggccagcccctacaccccagagcacgccgccagcgtgcccacccactcgccctacgcacaacccag
ctccaccttcgacaccatgtcgccggcgcctgtcatcccctccaacaccgactaccccggaccccaccactttgagg
tcactttccagcagtccagcacggccaagtcagccacctggacgtactccccgctcttgaagaaactctactgccag
atcgccaagacatgccccatccagatcaaggtgtccaccccgccacccccaggcaccgccatccgggccatgcctgt
ttacaagaaagcggagcacgtgaccgacgtcgtgaaacgctgccccaaccacgagctcgggagggacttcaacgaag
gacagtctgctccagccagccacctcatccgcgtggaaggcaataatctctcgcagtatgtggatgaccctgtcacc
ggcaggcagagcgtcgtggtgccctatgagccaccacaggtggggacggaattcaccaccatcctgtacaacttcat
gtgtaacagcagctgtgtagggggcatgaaccggcggcccatcctcatcatcatcaccctggagatgcgggatgggc
aggtgctgggccgccggtcctttgagggccgcatctgcgcctgtcctggccgcgaccgaaaagctgatgaggaccac
taccgggagcagcaggccctgaacgagagctccgccaagaacggggccgccagcaagcgtgccttcaagcagagccc
ccctgccgtccccgcccttggtgccggtgtgaagaagcggcggcatggagacgaggacacgtactaccttcaggtgc
gaggccgggagaactttgagatcctgatgaagctgaaagagagcctggagctgatggagttggtgccgcagccactg
gtggactcctatcggcagcagcagcagctcctacagaggccgacctgggggccctga (SEQ ID NO: 19)
Amino acid sequence (403 amino acids) NP_001191115.1
MAQSTATSPDGGTTFEHLWSSLEPDSTYFDLPQSSRGNNEVVGGTDSSMDVFHLEGMTTS
VMAQFNLLSSTMDQMSSRAASASPYTPEHAASVPTHSPYAQPSSTFDTMSPAPVIPSNTD
YPGPHHFEVTFQQSSTAKSATWTYSPLLKKLYCQIAKTCPIQIKVSTPPPPGTAIRAMPV
YKKAEHVTDVVKRCPNHELGRDFNEGQSAPASHLIRVEGNNLSQYVDDPVTGRQSVVVPY
EPPQVGTEFTTILYNFMCNSSCVGGMNRRPILIIITLEMRDGQVLGRRSFEGRICACPGR
DRKADEDHYREQQALNESSAKNGAASKRAFKQSPPAVPALGAGVKKRRHGDEDTYYLQVR
GRENFEILMKLKESLELMELVPQPLVDSYRQQQQLLQRPTWGP (SEQ ID NO: 20)
TAp73 epsilon
TAp73 epsilon CDS
LOCUS NM_001204187 4911 bp mRNA linear PRI 06-AUG-2019
DEFINITION Homo sapiens tumor protein p73 (TP73), transcript variant 11, mRNA.
ACCESSION NM_001204187
VERSION NM_001204187.1
atggcccagtccaccgccacctcccctgatgggggcaccacgtttgagcacctctggagctctctggaaccagacag
cacctacttcgaccttccccagtcaagccgggggaataatgaggtggtgggcggaacggattccagcatggacgtct
tccacctggagggcatgactacatctgtcatggcccagttcaatctgctgagcagcaccatggaccagatgagcagc
cgcgcggcctcggccagcccctacaccccagagcacgccgccagcgtgcccacccactcgccctacgcacaacccag
ctccaccttcgacaccatgtcgccggcgcctgtcatcccctccaacaccgactaccccggaccccaccactttgagg
tcactttccagcagtccagcacggccaagtcagccacctggacgtactccccgctcttgaagaaactctactgccag
atcgccaagacatgccccatccagatcaaggtgtccaccccgccacccccaggcaccgccatccgggccatgcctgt
ttacaagaaagcggagcacgtgaccgacgtcgtgaaacgctgccccaaccacgagctcgggagggacttcaacgaag
gacagtctgctccagccagccacctcatccgcgtggaaggcaataatctctcgcagtatgtggatgaccctgtcacc
ggcaggcagagcgtcgtggtgccctatgagccaccacaggtggggacggaattcaccaccatcctgtacaacttcat
gtgtaacagcagctgtgtagggggcatgaaccggcggcccatcctcatcatcatcaccctggagatgcgggatgggc
aggtgctgggccgccggtcctttgagggccgcatctgcgcctgtcctggccgcgaccgaaaagctgatgaggaccac
taccgggagcagcaggccctgaacgagagctccgccaagaacggggccgccagcaagcgtgccttcaagcagagccc
ccctgccgtccccgcccttggtgccggtgtgaagaagcggcggcatggagacgaggacacgtactaccttcaggtgc
gaggccgggagaactttgagatcctgatgaagctgaaagagagcctggagctgatggagttggtgccgcagccactg
gtggactcctatcggcagcagcagcagctcctacagaggccgccccgggatgctcaacaaccatggccacgcagtgc
cagccaacggcgagatgagcagcagccacagcgcccagtccatggtctcggggtcccactgcactccgccacccccc
taccacgccgaccccagcctcgtcaggacctgggggccctgaagatccccgagcagtaccgcatgaccatctggcgg
ggcctgcaggacctgaagcagggccacgactacagcaccgcgcagcagctgctccgctctagcaacgcggccaccat
ctccatcggcggctcaggggaactgcagcgccagcgggtcatggaggccgtgcacttccgcgtgcgccacaccatca
ccatccccaaccgcggcggcccaggcggcggccctgacgagtgggcggacttcggcttcgacctgcccgactgcaag
gcccgcaagcagcccatcaaggaggagttcacggaggccgagatccactga (SEQ ID NO: 21)
Amino acid sequence (555 amino acids) NP_001191116.1
MAQSTATSPDGGTTFEHLWSSLEPDSTYFDLPQSSRGNNEVVGGTDSSMDVFHLEGMTTS
VMAQFNLLSSTMDQMSSRAASASPYTPEHAASVPTHSPYAQPSSTFDTMSPAPVIPSNTD
YPGPHHFEVTFQQSSTAKSATWTYSPLLKKLYCQIAKTCPIQIKVSTPPPPGTAIRAMPV
YKKAEHVTDVVKRCPNHELGRDFNEGQSAPASHLIRVEGNNLSQYVDDPVTGRQSVVVPY
EPPQVGTEFTTILYNFMCNSSCVGGMNRRPILIIITLEMRDGQVLGRRSFEGRICACPGR
DRKADEDHYREQQALNESSAKNGAASKRAFKQSPPAVPALGAGVKKRRHGDEDTYYLQVR
GRENFEILMKLKESLELMELVPQPLVDSYRQQQQLLQRPPRDAQQPWPRSASQQRRDEQQ
PQRPVHGLGVPLHSATPLPRRPQPRDLGALKIPEQYRMTIWRGLQDLKQGHDYSTAQQLL
RSSNAATISIGGSGELQRQRVMEAVHFRVRHTITIPNRGGPGGGPDEWADFGFDLPDCKA
RKQPIKEEFTEAEIH (SEQ ID NO: 22)
TAp73 zeta
TAp73 zeta CDS
LOCUS NM_001204188 4866 bp mRNA linear PRI 06-AUG-2019
DEFINITION Homo sapiens tumor protein p73 (TP73), transcript variant 12, mRNA.
ACCESSION NM_001204188
VERSION NM_001204188.1
atggcccagtccaccgccacctcccctgatgggggcaccacgtttgagcacctctggagctctctggaaccagacag
cacctacttcgaccttccccagtcaagccgggggaataatgaggtggtgggcggaacggattccagcatggacgtct
tccacctggagggcatgactacatctgtcatggcccagttcaatctgctgagcagcaccatggaccagatgagcagc
cgcgcggcctcggccagcccctacaccccagagcacgccgccagcgtgcccacccactcgccctacgcacaacccag
ctccaccttcgacaccatgtcgccggcgcctgtcatcccctccaacaccgactaccccggaccccaccactttgagg
tcactttccagcagtccagcacggccaagtcagccacctggacgtactccccgctcttgaagaaactctactgccag
atcgccaagacatgccccatccagatcaaggtgtccaccccgccacccccaggcaccgccatccgggccatgcctgt
ttacaagaaagcggagcacgtgaccgacgtcgtgaaacgctgccccaaccacgagctcgggagggacttcaacgaag
gacagtctgctccagccagccacctcatccgcgtggaaggcaataatctctcgcagtatgtggatgaccctgtcacc
ggcaggcagagcgtcgtggtgccctatgagccaccacaggtggggacggaattcaccaccatcctgtacaacttcat
gtgtaacagcagctgtgtagggggcatgaaccggcggcccatcctcatcatcatcaccctggagatgcgggatgggc
aggtgctgggccgccggtcctttgagggccgcatctgcgcctgtcctggccgcgaccgaaaagctgatgaggaccac
taccgggagcagcaggccctgaacgagagctccgccaagaacggggccgccagcaagcgtgccttcaagcagagccc
ccctgccgtccccgcccttggtgccggtgtgaagaagcggcggcatggagacgaggacacgtactaccttcaggtgc
gaggccgggagaactttgagatcctgatgaagctgaaagagagcctggagctgatggagttggtgccgcagccactg
gtggactcctatcggcagcagcagcagctcctacagaggccttttttaacaggattggggtgtccaaactgcatcga
gtatttcacctcccaagggttacagagcatttaccacctgcagaacctgaccattgaggacctgggggccctgaaga
tccccgagcagtaccgcatgaccatctggcggggcctgcaggacctgaagcagggccacgactacagcaccgcgcag
cagctgctccgctctagcaacgcggccaccatctccatcggcggctcaggggaactgcagcgccagcgggtcatgga
ggccgtgcacttccgcgtgcgccacaccatcaccatccccaaccgcggcggcccaggcggcggccctgacgagtggg
cggacttcggcttcgacctgcccgactgcaaggcccgcaagcagcccatcaaggaggagttcacggaggccgagatc
cactga (SEQ ID NO: 23)
Amino acid sequence (540 amino acids) NP_001191117.1
MAQSTATSPDGGTTFEHLWSSLEPDSTYFDLPQSSRGNNEWGGTDSSMDVFHLEGMTTS
VMAQFNLLSSTMDQMSSRAASASPYTPEHAASVPTHSPYAQPSSTFDTMSPAPVIPSNTD
YPGPHHFEVTFQQSSTAKSATWTYSPLLKKLYCQIAKTCPIQIKVSTPPPPGTAIRAMPV
YKKAEHVTDVVKRCPNHELGRDFNEGQSAPASHLIRVEGNNLSQYVDDPVTGRQSVVVPY
EPPQVGTEFTTILYNFMCNSSCVGGMNRRPILIIITLEMRDGQVLGRRSFEGRICACPGR
DRKADEDHYREQQALNESSAKNGAASKRAFKQSPPAVPALGAGVKKRRHGDEDTYYLQVR
GRENFEILMKLKESLELMELVPQPLVDSYRQQQQLLQRPFLTGLGCPNCIEYFTSQGLQS
IYHLQNLTIEDLGALKIPEQYRMTIWRGLQDLKQGHDYSTAQQLLRSSNAATISIGGSGE
LQRQRVMEAVHFRVRHTITIPNRGGPGGGPDEWADFGFDLPDCKARKQPIKEEFTEAEIH (SEQ ID NO: 24)

In particular embodiments, the agent that targets p73 or an isoform thereof is a nucleic acid that targets a sequence provided in SEQ ID NOS:1, 3, 5, 7, 9, 11, 13, 14, 15, 16, 17, 18, 19, 21, 22, 23, or 24. The targeted sequence may be of any length in the polynucleotide such as at least the following lengths of nucleotides or a targeted sequence of at least about the following lengths of nucleotides (nt): 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 125, 150, 175, 200, 300, 400, 500, or more. The length of the nucleic acid agent that targets the p73 polynucleotide or isoform polynucleotide polynucleotide may of any length including at least the following lengths of nt or at least about the following lengths of nt: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 125, 150, 175, 200, 300, 400, 500, or more.

In certain embodiments, the agent that targets p73 or an isoform thereof is a protein or small molecule that targets a protein of SEQ ID NOS:2, 4, 6, 8, 10, 12, 14, 16, 18, 20, 22, or 24.

### IV. Methods of Treatment

Methods of treating cancer are encompassed herein. Embodiments of the disclosure encompass methods of treating an individual for cancer that is resistant to at least one therapy.

Provided herein are methods of treating cancer in a subject undergoing treatment with one or more BH3 mimetics and/or that has undergone treatment with one or more BH3 mimetics. The methods include administering to the subject an anti-cancer resistance agent that is an agent that targets p73 or an isoform thereof. In some embodiments, the cancer is a BH3 mimetic-resistant cancer. In some embodiments, acquired cancer resistance to the BH3 mimetic is decreased or prevented with methods of the disclosure. In some embodiments, the activity of the BH3 mimetic is directly or indirectly increased with methods of the disclosure.

Provided herein are methods for preventing (or reducing the extent of or reducing the risk of) cancer resistance or enhancing the therapeutic efficacy of one or more BH3 mimetics in a subject having cancer and undergoing treatment with one or more BH3 mimetics. The methods may include administering to the subject a pharmaceutical composition comprising one or more anti-cancer resistance agents in an amount effective to attenuate cancer resistance to the BH3 mimetic(s). In some embodiments, acquired cancer resistance to the BH3 mimetic(s) is decreased or prevented or delayed in onset. In some embodiments, the activity of the BH3 mimetic(s) is directly or indirectly increased.

Provided herein are methods of sensitizing a BH3-resistant cancer in a subject to one or more BH3 mimetics. The methods may include administering to the subject one or more anti-cancer resistance agents. In some embodiments, acquired cancer resistance to the BH3 mimetic(s) is decreased or prevented or the onset is delayed. In some embodiments, the activity of the BH3 mimetic(s) is increased. In some embodiments, one or more agents that target p73 or an isoform thereof can be used to sensitize BH3 mimetic-resistant cancer cells.

In particular embodiments, the methods and compositions employed for such methods allow a cancer that is resistant to a first therapy to be able to be treated as a result of employing a second therapy that overcomes the cancer resistance to the first therapy. In specific embodiments, the disclosure provides methods of rendering a therapy effective for a cancer that is resistant to it by treating with a second therapy that targets the cause of the cancer resistance or that targets a factor associated with the cancer being resistant.

Certain embodiments of the disclosure concern reducing cancer resistance to an anti-apoptotic protein therapy by administering an additional therapy that targets a gene product associated with the cancer resistance (such as becoming detectable upon development of cancer resistance).

In some cases, the BH3 mimetic resistance develops over time, such as over the course of being treated with the BH3 mimetic(s), and in other cases the cancer of the individual is resistant from the first treatment with the BH3 mimetic(s).

In particular embodiments, the disclosure concerns reducing cancer resistance to BH3 mimetic(s) so that the BH3 mimetic(s) can be effective in cancer of an individual that is resistant to the BH3 mimetic(s). The cancer resistance may be reduced entirely (no longer detectable, for example) or partially, and in cases where the cancer resistance is reduced partially, the individual may or may not be in need of a greater concentration of the BH3 mimetic for efficacy that is higher than the concentration utilized when the cancer resistance is entirely reduced.

In specific embodiments, the disclosure provides methods of treating an individual for a BH3 mimetic-resistant cancer by allowing the BH3 mimetic(s) to be effective upon overcoming cancer resistance to the BH3 mimetic(s).

In particular embodiments, there are methods of treating cancer in an individual comprising the step of providing to the individual an effective amount each of the following: (1) one or more BH3 mimetics; and (2) one or more agents that target p73 or an isoform thereof.

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are particular to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In some embodiments, methods of the disclosure encompass a step of identifying whether or not cancer of an individual is resistant to BH3 mimetics or will become resistant to BH3 mimetics. The disclosure includes methods that treat an individual with an agent that targets p73 or an isoform thereof when the individual has been identified to have p73 or an isoform thereof, including TAp73 and/or ΔNp73. The determination of p73 or an isoform thereof may be performed by any suitable method, and the determination may include assaying for nucleic acid and/or protein. For example, TAp73 and/or ΔNp73 protein levels may be determined by western blot, and TAp73 and/or ΔNp73 mRNA levels may be determined by PCR.

### V. Pharmaceutical Compositions

Pharmaceutical compositions that may be utilized in methods of treating cancer may comprise one or more BH3 mimetics and/or one or more agents that target p73 or an isoform thereof and/or one or more p53 activators. In some embodiments, the one or more BH3 mimetics are inhibitors of Bcl-2, Bcl-xL, Mcl-1, Bcl-W, Al/BFL-1, or a mixture thereof. Examples of mixtures of inhibitors include a mixture of Bcl-2 inhibitor and Mcl-1 inhibitor, or a mixture of Bcl-xL inhibitor and Bcl-2 inhibitor. In specific embodiments, a pharmaceutical composition comprises one or more activators of p53. In cases where the BH3 mimetics and/or one or more agents that target p73 or an isoform thereof and/or one or more activators of p53 are administered together, they may be stored or transported in the same composition or in different compositions. In cases where the BH3 mimetics and/or one or more agents that target p73 or an isoform thereof and/or one or more activators of p53 are not administered together, they may be stored or transported in different compositions.

Pharmaceutical compositions of the present disclosure comprise an effective amount of one or more BH3 mimetics and/or one or more agents that target p73 or an isoform thereof and/or one or more activators of p53, each dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that comprises at least one BH3 mimetics and/or one or more agents that target p73 or an isoform thereof and/or one or more activators of p53 or additional active ingredient will be known to those of skill in the art in light of the present disclosure, as exemplified by Remington: The Science and Practice of Pharmacy, 21^{st} Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The BH3 mimetic(s) and/or one or more agents that target p73 or an isoform thereof and/or one or more activators of p53 may comprise different types of carriers depending on whether it is to be administered in solid, liquid or aerosol form, and whether it need to be sterile for such routes of administration as injection. The pharmaceutical compositions can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in creams, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The BH3 mimetics and/or one or more agents that target p73 or an isoform thereof and/or one or more activators of p53 may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present disclosure, the compositions of the present disclosure suitable for administration are provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present disclosure, the composition may be combined with the carrier in any convenient and practical manner, i.e., by solution, suspension, emulsification, admixture, encapsulation, absorption and the like. Such procedures are routine for those skilled in the art.

In a specific embodiment of the present disclosure, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present disclosure may concern the use of a pharmaceutical lipid vehicle compositions that include BH3 mimetics and/or one or more agents that target p73 or an isoform thereof, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the BH3 mimetics and/or one or more agents that target p73 or an isoform thereof and/or one or more activators of p53 may be dispersed in a solution comprising a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present disclosure administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a particular dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein. In specific cases, the BH3 mimetics and/or one or more agents that target p73 or an isoform thereof and/or one or more activators of p53 may comprise about 0.1, 0.2, 0.3, 0.4, 0.5, 0.75, 0.8, 0.9, 1, 2, 3, 4, 5, 6, 7, 8, 9, 10, 12, 15, 18, 20, 25, 30, 35, 40, 45, 50, 55, 65, 70, 75, 80, 85, 90, 95, 97, or 99%, or more of the weight of the unit. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

C. Alimentary Compositions and Formulations

In particular embodiments of the present disclosure, the BH3 mimetics and/or one or more agents that target p73 or an isoform thereof and/or one or more activators of p53 are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (U.S. Pat. Nos. 5,641,515; 5,580,579 and 5,792,451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present disclosure may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations that are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

D. Parenteral Compositions and Formulations

In further embodiments, BH3 mimetics and/or one or more agents that target p73 or an isoform thereof and/or one or more activators of p53 may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,613,308; 5,466,468; 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

E. Miscellaneous Pharmaceutical Compositions and Formulations

In other particular embodiments of the disclosure, the active compound BH3 mimetics and/or one or more agents that target p73 or an isoform thereof and/or one or more activators of p53 may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins and lysophosphatidyl-glycerol compounds (U.S. Pat. No. 5,725, 871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### VI. Kits

Any of the compositions described herein may be comprised in a kit. In a non-limiting example, one or more BH3 mimetics and/or one or more agents that target p73 or an isoform thereof and/or one or more activators of p53 may be comprised in a kit. The kits will thus comprise, in suitable container means, one or more BH3 mimetics and/or one or more agents that target p73 or an isoform thereof and/or one or more activators of p53.

The kits may comprise compositions comprising a suitably aliquoted BH3 mimetic(s) and/or agent(s) that target p73 or an isoform thereof. The BH3 mimetic(s) and the agent(s) that target p73 or an isoform thereof may or may not be comprised in the same composition in the kit. The BH3 mimetic(s) and agent(s) that target p73 or an isoform thereof may be housed separately in the kit and may be configured to be combined prior to administration to an individual.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one components in the kit, the kit also may generally contain a second, third or other additional container into which the additional components may be separately placed. However, various combinations of components may be comprised in a vial. The kits of the present disclosure also will typically include a means for containing the BH3 mimetic(s) and/or agents that target p73 or an isoform thereof, and any other reagent containers, in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. The compositions may also be formulated into a syringeable composition. In such a case, the container means may itself be a syringe, pipette, and/or other such like apparatus, from which the formulation may be applied to an affected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

Irrespective of the number and/or type of containers, the kits of the disclosure may also comprise, and/or be packaged with, an instrument for assisting with the injection/administration and/or placement of the ultimate composition within the body of an animal. Such an instrument may be a syringe, pipette, forceps, and/or any such medically approved delivery vehicle.

### EXAMPLES

The following examples are included to demonstrate particular embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### TP73 Isoforms (TAp73 And ΔNp73) are Overexpressed in Acute Myeloid Leukemias and Potential Therapeutic Targets to Enhance Anti-Leukemia Activities of Bcl-2 and MDM2 Inhibitors

Background TP73 is one of the TP53 family transcription factors and generates two isoforms, the transactivation p73 (TAp73) and the N-terminally truncated ΔNp73. TAp73 shares a homologous N-terminal activation domain with p53 and has similar pro-apoptotic function to p53. ΔNp73 lacks an activation domain and has activities distinct from TAp73. ΔNp73 has a dominant negative effect on the DNA binding of TAp73 and more importantly, of p53, since the DNA binding domain is homologous with that of TAp73 and highly similar to that of p53. In acute myeloid leukemias (AML), TP73 has been reported to be expressed except in acute promyelocytic leukemias. However, the association of TP73 isoforms with clinical and genetic characteristics and the regulation of the isoforms in AML have not been explored.

Results The inventors determined copy numbers of ΔNp73 and TAp73 mRNA levels in 78 AML samples including 31 de novo AML using droplet digital PCR (ddPCR), which allows to determine the absolute quantity of the isoforms expressed, and investigated their clinical and biological relevance. ΔNp73 and TAp73 expression was detected in 93.6% and 98.7% of AML samples at variable levels (mean±SEM, 10.6±5.0, and 106.6±33.7 copies/μL, for ΔNp73 and TAp73, respectively). ΔNp73 and TAp73 mRNA levels were highly correlated (R²=0.72, P<0.0001). Normal peripheral blood mononuclear cells and CD34+ hematopoietic cells showed little or no ΔNp73 and TAp73 expression (0.09±0.09 and 0.42±0.35 copies/μL, respectively), demonstrating that expression of ΔNp73 and TAp73 is 100-1,000 fold higher in AML as compared to normal hematopoietic cells. These data collectively suggests that transcriptional systems of both isoforms in AML cells are abnormally activated. Disease status (de novo or relapsed/refractory) and cytogenetic abnormalities did not correlate with ΔNp73 and TAp73 levels. However, AML with IDH1/2 mutations had 8.5-fold lower ΔNp73 expression than those with wild-type IDH1/2 (1.8±0.8 vs 15.4±7.7 copies/μL, P=0.0069), with a similar trend for TAp73 (49.0±20.3 vs 138.6±51.4 copies/μL, P=0.056). For de novo AML samples, those with DNMT3a and NRAS mutations had significantly higher ΔNp73, but not TAp73, than those without these mutations (21.6±18.2 vs 2.5±1.2 copies/μL, P=0.017 and 5.6±2.5 vs 9.7±8.0 copies/μL, P=0.047, respectively). These findings suggest that ΔNp73 and TAp73 can be differentially regulated in AML based on mutation status. To further explore the regulation of TP73 isoforms, the inventors used MDM2 inhibitor Nutlin-3a to induce p53 which is a transcriptional inducer of ΔNp73. Indeed, MDM2 inhibition increased p73 protein levels, and knockdown of both TAp73 and ΔNp73 in AML cells enhanced apoptosis induction by Nutlin-3a (specific annexin V induction by 5 μM Nutlin-3a, 21.9±1.3% vs 61.3±5.2%, P=0.0084 in OCI-AML3 cells with vector control vs Shp73, respectively), possibly due to the silencing of ΔNp73. AML cells with IDH1/2 mutations are more dependent on Bcl-2 than those without (Chan, Nat Med 2015). Intriguingly, (R)-2HG, the oncometabolite of mutant IDH1/2, reduced both TAp73 and ΔNp73 in AML cells and increased susceptibility to the Bcl-2 inhibitor ABT-199. These results imply a potential mechanism that regulates p73 isoforms by histone methylation or other epigenetic modifications in AML.

Conclusion Absolute quantitation of TP73 isoforms by ddPCR revealed high expression in AML cells compared to normal hematopoietic cells. The repressed expression of TP73 isoforms in AML cells with IDH1/2 mutations or by the oncometabolite (R)-2HG suggests that epigenetic modifications through (R)-2HG can regulate TP73 transcription and enhance the anti-leukemia effect by Bcl-2 inhibition. Finally, downregulation of TP73 isoforms enhances the efficacy of MDM2 inhibitor in AML, suggesting a potential therapeutic strategy to enhance MDM2 inhibitor-mediated p53 activation.

### example 2

TP73 as Novel Determinant of Resistance to Bcl-2 Inhibition in Acute Myeloid Leukemia

Background. BCL-2 inhibition is a novel and highly effective treatment modality in acute myeloid leukemias (AML). AML patients with IDH1/2 mutations are highly sensitive to BCL-2 inhibition by venetoclax (VEN) (Chen et al Nat Med 2015). High expression levels of the BCL-2 family proteins MCL-1 or BCL-X_{L}, or knockout of TP53 have been reported to confer resistance to BCL-2 inhibition (Pan et al. Cancer Cell 2017, Nechiporuk et al. Cancer Discov 2019). p73 is one of the p53 family transcription factors and generates two isoforms, transactivation p73 (TAp73) and the N-terminally truncated ΔNp73. TAp73 shares a homologous N-terminal activation domain with p53 and has pro-apoptotic function similar to p53. ΔNp73 lacks an activation domain and has a dominant negative effect on the DNA binding of TAp73 and more importantly, of p53. TP73 is expressed in AML except in acute promyelocytic leukemias. However, the associations of TP73 isoforms with clinical and genetic characteristics or sensitivity to BCL-2 inhibition in AML have not been explored.

Results. The inventors determined copy numbers of TAp73 and ΔNp73 mRNA levels in AML samples (N=78) and normal CD34+ hematopoietic cells (HPC) using droplet digital PCR and investigated their clinical and biological relevance. Both TP73 isoforms were expressed in AML, with TAp73 expression being 50-fold higher in AML than in CD34+ HPC (P=0.027); no difference seen for ΔNp73 (P=0.80), suggesting that TAp73 is aberrantly expressed in AML cells. ΔNp73 and TAp73 mRNA levels were highly correlated (R²=0.72, P<0.0001). AML samples had 10-fold more abundant TAp73 than ΔNp73 mRNA levels (P=0.0017) and isoforms were not associated with disease status (de novo vs relapsed/refractory) or cytogenetic groups, and were mutation-agnostic, except for IDH1/2. IDH1/2 mutant AML showed lower levels of TAp73 and ΔNp73 than those with wild-type IDH1/2 (P=0.06 and P=0.007 for TAp73 and ΔNp73 , respectively). In a separate dataset, there was repressed TP73 in IDH1/2 mutant vs. wild-type AML samples (P=0.073) by RNAseq analysis (N=47).

Mechanistically, treatment with cell permeable octyl-(R)-2HG, the oncometabolite of mutant IDH1/2, reduced both TAp73 and ΔNp73 and increased susceptibility to VEN. Lentiviral knockdown of p73 in OCI-AML3 cells resulted in enhanced sensitivity to VEN with no significant changes in MCL-1 and p53 protein levels, or TP53 targets (MDM2, CDKN 1A, FAS and BBC3). VEN resistant AML cells (MOLM-13 and MV4;11) generated through long-term culture with VEN expressed highly elevated TP73 mRNA and protein levels without significant changes in p53 or TP53 target changes, suggesting that elevated p73 could confer resistance to VEN independent of p53 function. Knockdown of TP73 showed increased protein levels of SDHB, UQCRC2 and ATP5A, components of mitochondrial respiratory chain complex II, III and V, indicating increased dependency on oxdative phosphorylation by depleting p73. Overexpression of TAp73α by lentiviral gene transfer minimally increased VEN-induced apoptosis, while ΔNp73γ overexpression conferred striking resistance to VEN in MOLM-13 cells, suggesting p73 isoform-specific dependency of VEN sensitivity/resistance. The combination of 5′-azacitidine (5′-aza) and VEN decreased ΔNp73 level by 50%.

Conclusion. Repression of TP73 in IDH1/2 mutant AML, and downregulation of TP73 by the oncometabolite 2-HG were associated with enhanced sensitivity to VEN, suggesting that TP73 determines AML susceptibility to BCL-2 inhibition. VEN resistant cells massively overexpressed TP73, and TP73 knockdown restored sensitivity to VEN. Specifically, overexpression of the ΔNp73γ isoform resulted in induced VEN resistance. ΔNp73 levels were also reduced by combining VEN with 5′-aza. Results may explain the high sensitivity of IDH1/2 mutant AML to VEN as consequence of downregulation of TP73 by 2-HG, and establish the mechanism of synergistic effect by VEN+5′-aza combination and overexpression of p73 as a novel resistance mechanism to BCL-2 inhibition.

### Example 3

### Targeting P73 for Cancers Resistant to BH3 Mimetics

The present examples demonstrates efficacy in targeting p73 for cancers that are resistant to particular examples of BH3 mimetics.

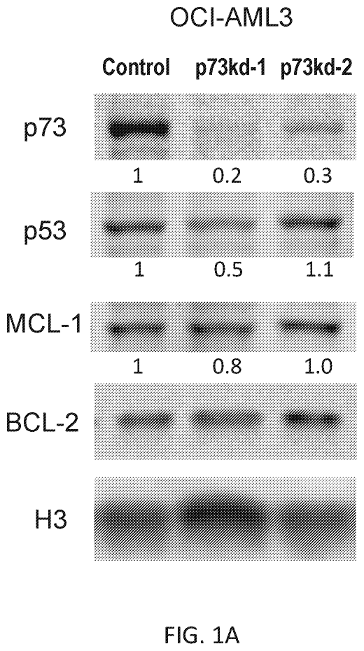

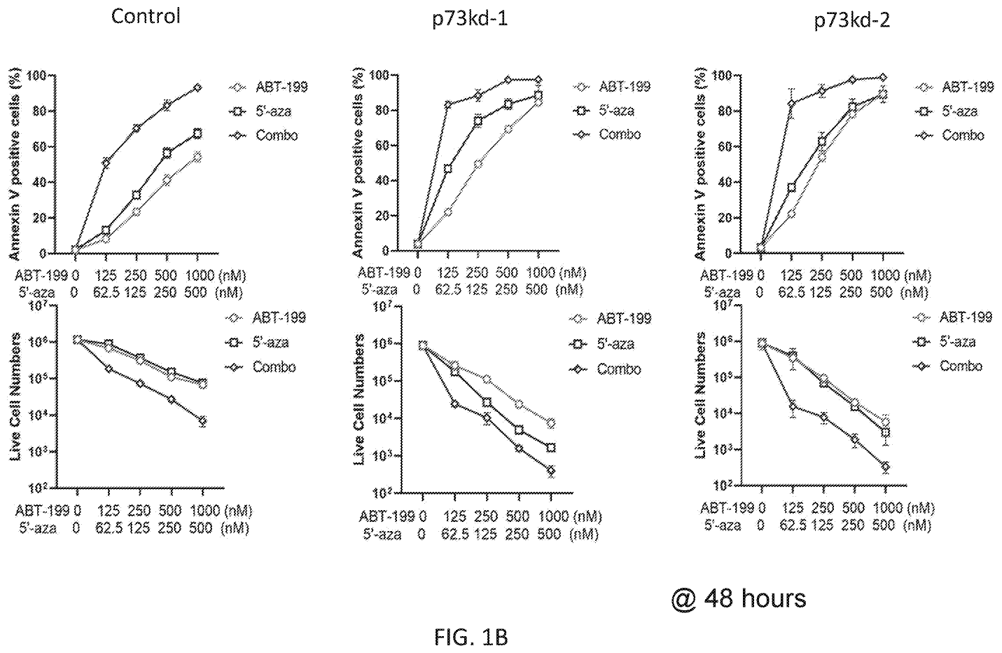

p73 knockdown enhances apoptosis induction by ABT-199 (venetoclax, catalogue No.S8048, CAS 1257044-40-8), 5′-azacitidine and combined treatment in a MCL-1 and BCL-2 independent manner. As shown in FIG. 1A, there are immunoblot images of OCI-AML3 cells with p73 knockdown (kd-1 and kd-2) by lentiviral transfection. Histone H3 (H3) serves as a loading control, and relative protein levels were measured as ratios of target proteins and H3 through densitometry. p73 knockdown did not affect protein levels of MCL-1 and BCL-2, which are known as determinants of sensitivity to BCL-2 inhibition (Pan et al. Cancer Discov 2014, Pan et al. Cancer Cell 2017).

In FIG. 1B, there is shown sensitivity to ABT-199, 5′-azacitidine (5-aza) (DNA demethylating agent, or hypomethylating agent (HMA)) and combined treatment of both drugs. The combination is now considered a new standard for the treatment of elderly patients with AML (DiNardo et al., Blood 2019). Apoptosis induction was measured by annexin V staining (Annexin V positive cells (%)), and live cell numbers were measured by counting beads by flow cytometry. In FIG. 1B, p73 knockdown demonstrated greater ABT-199- or ABT/5-aza combo-mediated apoptosis (upper panel) than control, and superior reduction of live cell numbers (lower panel) in OCI-AML3 cells.

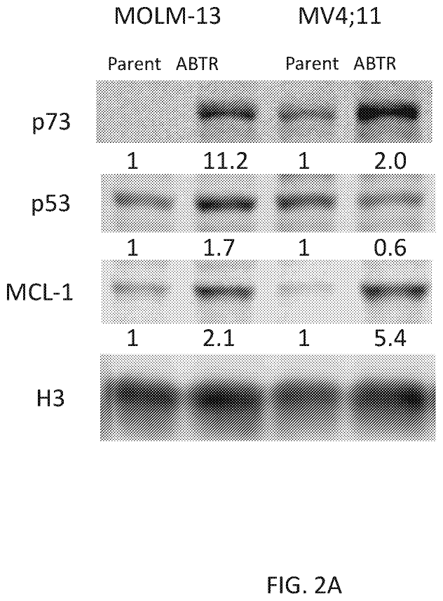

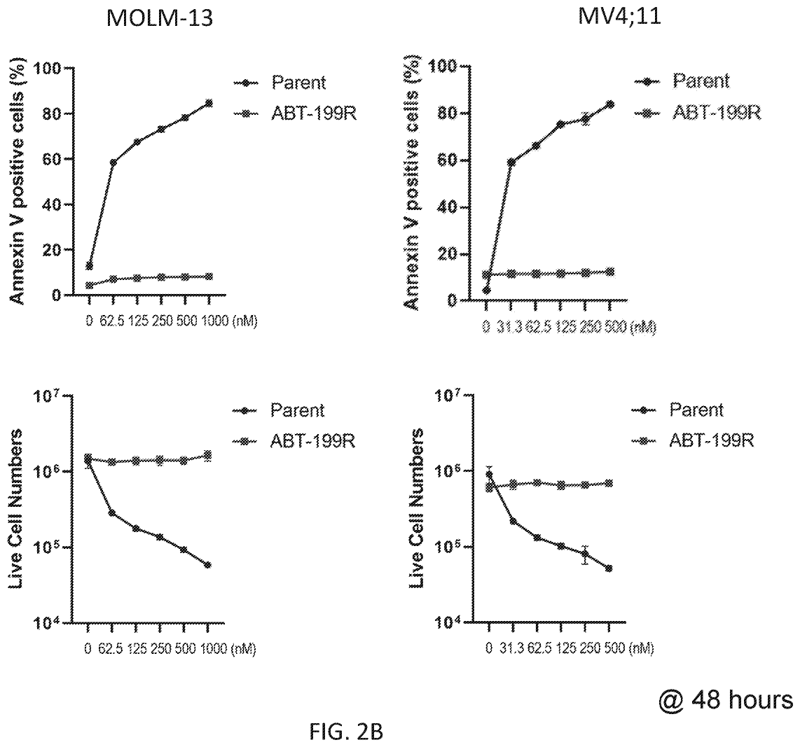

FIG. 2A-2B demonstrate that ABT-199 resistant AML cells show elevated p73 protein levels. In FIG. 2A, there are immunoblot images of two AML cell lines (MOLM-13 and MV4;11 cells) that acquired ABT-199 resistance (ABTR) through long-term exposure to continuous treatment of ABT-199. Parental control cells (Parent) are used for comparison. Histone H3 (H3) serves as a loading control, and relative protein levels were measured as ratios of target proteins and H3 through densitometry. MOLM-13 and MV4;11 ABTR cells showed elevated protein levels of MCL-1, which is evidence for MCL-1 as an ABT-199 resistance mechanism. However, it was also found that those cells have higher p73 protein levels. In FIG. 2B, there is shown sensitivity of MOLM-13 and MV4;11 parent and ABTR cells to ABT-199. Apoptosis induction was measured by annexin V staining (Annexin V positive cells (%)), and live cell numbers were measured by counting beads by flow cytometry. ABTR cells (lines with squares) clearly show resistance to ABT-199 (venetoclax).These results serve as a proof of ABT-199 resistance of those cells.

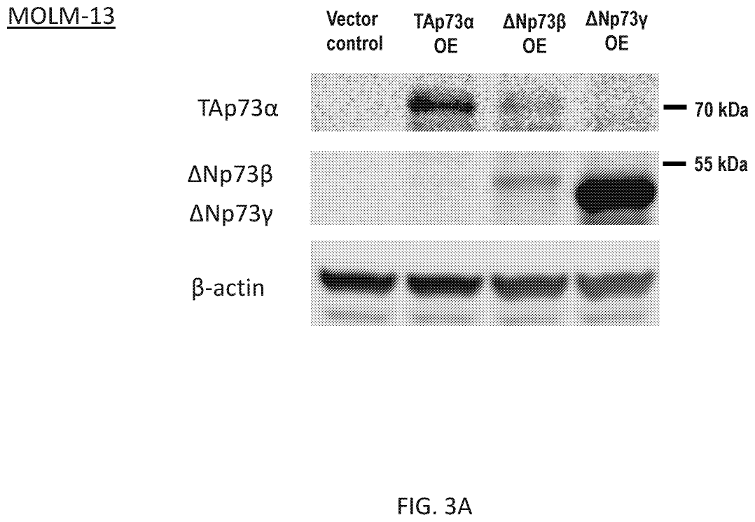

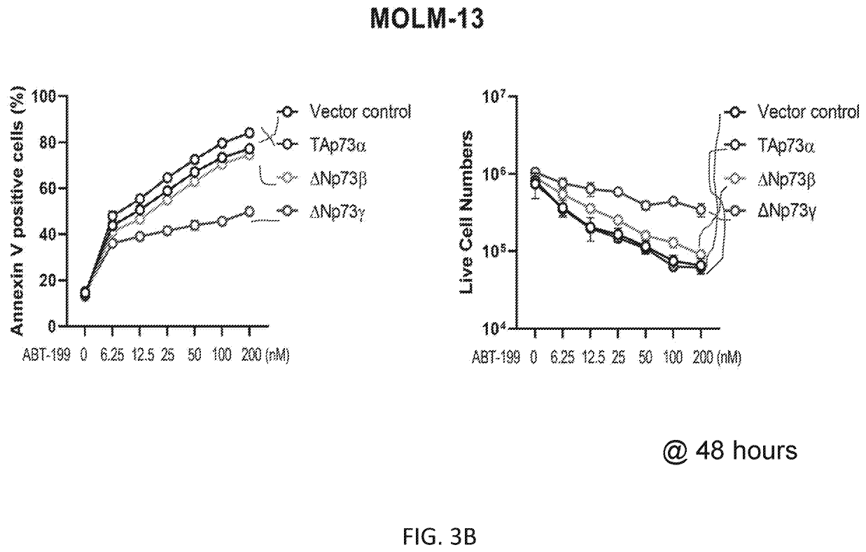

FIGS. 3A-3B show that overexpression of ΔNp73γ directly results in ABT-199 resistance. In FIG. 3A, there are immunoblot images of different p73 N-terminal isoforms (TA and ΔN) and C-terminal variants (α, β and γ) protein levels. MOLM13 cells with overexpression of those p73 isoforms/variants was generated by lentiviral transfection. β-actin serves as a loading control. Successful overexpression of p73 isoforms/variants in MOLM-13 cells, which are originally sensitive to ABT-199. In FIG. 3B, sensitivity to ABT-199 in MOLM-13 cells with overexpression of p73 isoforms/variants is demonstrated. Apoptosis induction was measured by annexin V staining (Annexin V positive cells (%)), and live cell numbers were measured by counting beads by flow cytometry. MOLM-13 cells with ΔNp73γ overexpression were resistant to ABT-199 whereas those with vector control and TAp73α and ΔNp73β overexpression were still sensitive to ABT-199. A p73 isoform ΔNp73γ overexpression induced resistance to ABT-199, indicating a novel resistance mechanism by p73 isoforms/variants to ABT-199 in AML cells.

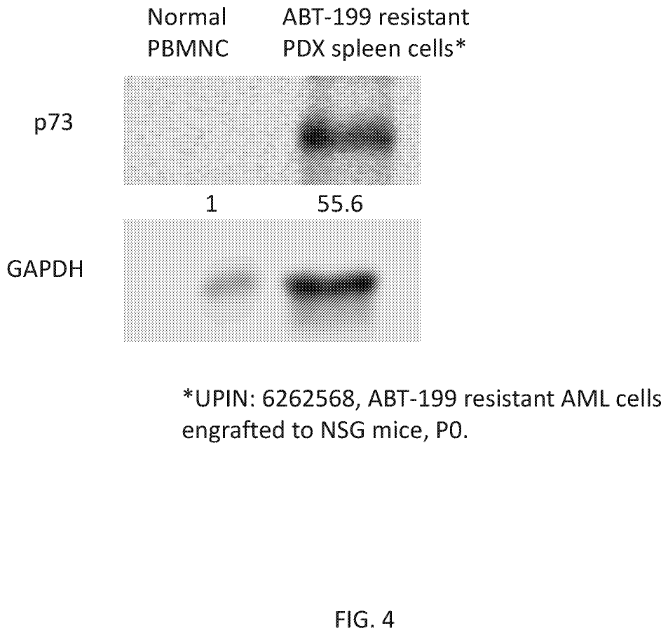

FIG. 4 provides immunoblot images of patient-derived xenograft (PDX) AML cells engrafted in NSG mice. Primary AML cells from a patient who showed venetoclax resistance were injected into NSG mice. The inventors sacrificed and dissected the mice to collect AML cells from their bone marrow, liver and spleen. Spleen cells were lysed for immunoblotting. GAPDH serves as a loading control, and relative protein levels were measured as ratios of target proteins and GAPDH through densitometry. PDX AML cells with venetoclax resistance show elevated p73 protein levels compared to normal bone marrow hematopoietic cells, which supports the finding in FIG. 2.

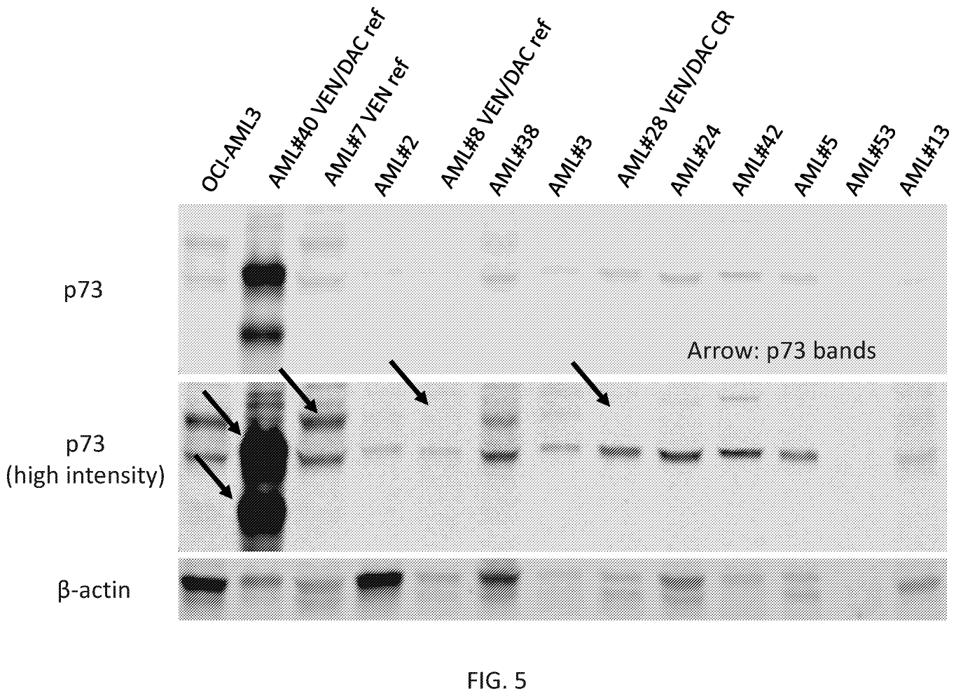

FIG. 5 demonstrates that patient-derived (primary) AML samples with venetoclax (VEN) resistance show high p73 protein levels. Immunoblot images of primary AML samples for p73 protein levels are provided. The middle panel was an intensified image of the top panel (same image with high intensity). Three VEN-resistant (AML #40, #7 and #8) and one VEN-sensitive (AML #28) samples are shown. Arrows highlight p73 bands. Primary AML cells with VEN resistance show high or visible p73 protein levels while a VEN sensitive sample show no band of p73.

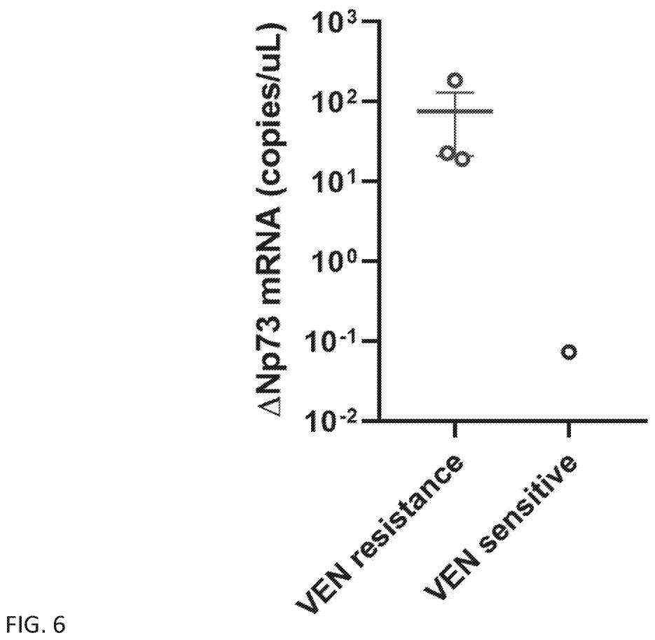

FIG. 6 indicates that venetoclax (VEN)-resistant primary AML cells show higher ΔNp73 mRNA levels than a VEN sensitive AML sample. ΔNp73 transcript levels of VEN resistant and sensitive primary AML cells were measured by droplet digital PCR. VEN resistant primary AML cells show higher ΔNp73 mRNA levels than a VEN sensitive AML sample, supporting the finding of FIGS. 3 and 5.

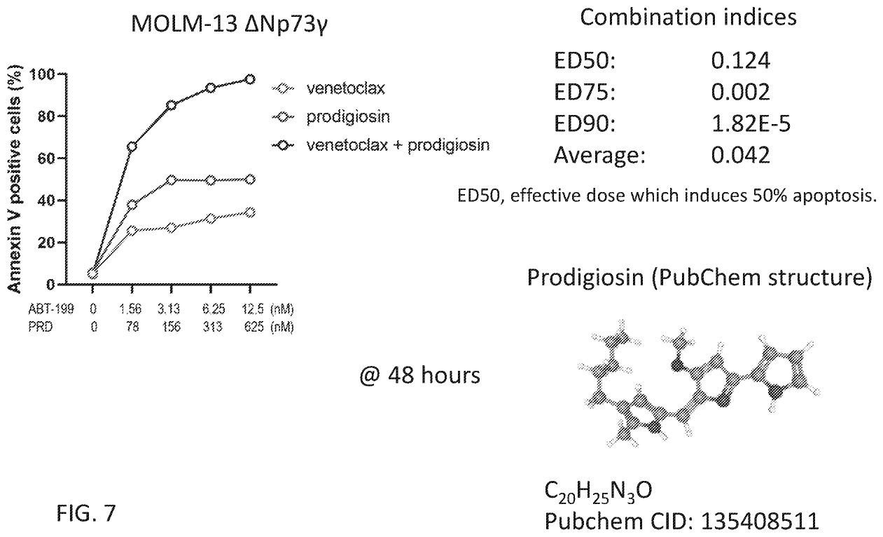

In FIG. 7, apoptosis induction was measured by annexin V staining (Annexin V positive cells (%)), and live cell numbers were measured by counting beads by flow cytometry. Combinatorial treatment of ABT-199 (venetoclax) with prodigiosin induces synergistic apoptosis in MOLM-13 ΔNp73γ-overexpres sing cells.

### REFERENCES

All publications mentioned in this specification are indicative of the level of those skilled in the art to which the invention pertains. All publications herein are incorporated by reference to the same extent as if each individual publication was specifically and individually indicated to be incorporated by reference in their entirety.
U.S. Pat. No. 5,580,579
U.S. Pat. No. 5,641,515
U.S. Pat. No. 5,725,871
U.S. Pat. No. 5,756,353
U.S. Pat. No. 5,780,045
U.S. Pat. No. 5,792,451
U.S. Pat. No. 5,804,212

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A composition comprising:
(a) one or more BH3 mimetics; and
(b) one or more agents that target p73 or an isoform thereof.

2. The composition of claim 1, further comprising (c) one or more activators of p53.

3. The composition of claim 1 or 2, wherein the composition is comprised in a pharmaceutically acceptable excipient.

4. The composition of any one of claims 1-3, wherein (a) and/or (b) and/or (c) are housed in separate containers.

5. The composition of any one of claims 1-3, wherein (a) and/or (b) and/or (c) are housed in the same container.

6. The composition of any one of claims 1-5, wherein the BH3 mimetic is an inhibitor of Bcl-2, Bcl-xL, Mcl-1, Bcl-W, A1/BFL-1, or a mixture thereof.

7. The composition of any one of claims 1-6, wherein the BH3 mimetic is a combination of an inhibitor of Bcl-2 and an inhibitor of Bcl-xL.

8. The composition of any one of claims 1-6, wherein the BH3 mimetic is a combination of an inhibitor of Bcl-2 and an inhibitor of Mcl-1.

9. The composition of any one of claims 1-8, wherein the BH3 mimetic is not a Bcl-2 inhibitor.

10. The composition of any one of claims 1-9, wherein the BH3 mimetic is AZD5991, ABT-737, ABT-199 (Venetoclax), ABT-263 (Navitoclax), or a mixture thereof.

11. The composition of any one of claims 1-10, wherein the agent that targets p73 or an isoform thereof comprises a small molecule, protein, nucleic acid, or combination or mixture thereof.

12. The composition of any one of claims 1-11, wherein the agent that targets p73 or an isoform thereof is a small molecule.

13. The composition of claim 12, wherein the small molecule is octyl-(R)-2HG, octyl-(L)-2HG, or a mixture thereof.

14. The composition of claim 11, wherein the nucleic acid is a shRNA or CRISPR molecule.

15. The composition of claim 11, wherein the protein is an antibody.

16. The composition of any one of claims 1-15, wherein the composition is comprised in a kit.

17. A method of treating cancer, comprising the step of administering to an individual with cancer a therapeutically effective amount of the composition of any one of claims 1-16.

18. The method of claim 17, wherein the cancer is resistant to one or more BH3 mimetics.

19. The method of claim 17 or 18, wherein in the composition the one or more BH3 mimetics and the one or more agents that target p73 or an isoform thereof are administered to the individual at the same time.

20. The method of any one of claims 17-19, wherein the one or more BH3 mimetics and the one or more agents that target p73 or an isoform thereof are administered to the individual in the same formulation.

21. The method of any one of claim 17 or 18, wherein the one or more BH3 mimetics and the one or more agents that target p73 or an isoform thereof are administered to the individual at different times.

22. The method of claim 21, wherein the one or more BH3 mimetics are administered to the individual prior to the one or more agents that target p73 or an isoform thereof.

23. The method of claim 21, wherein the one or more BH3 mimetics are administered to the individual subsequent to the one or more agents that target p73 or an isoform thereof.

24. The method of any one of claims 17-23, wherein the cancer is a hematological cancer or comprises a solid tumor.

25. The method of any one of claims 17-24, wherein the cancer is chronic lymphocytic leukemia, acute myeloid leukemia, acute lymphocytic leukemia, chronic myeloid leukemia, Hodgkin's lymphoma, non-Hodgkin's lymphoma, or myeloma.

26. The method of any one of claims 17-25, wherein the p73 isoform is transactivation p73 (Tap73) or ΔNp73.

27. The method of any one of claims 17-26, wherein the agent that targets p73 or an isoform thereof comprises a small molecule, protein, nucleic acid, or combination or mixture thereof.

28. The method of any one of claims 17-27, wherein the agent that targets p73 or an isoform thereof is a small molecule.

29. The method of claim 28, wherein the small molecule is octyl-(R)-2HG, octyl-(L)-2HG, or a mixture thereof.

30. The method of claim 27, wherein the nucleic acid is a shRNA or CRISPR molecule.

31. The method of claim 27, wherein the protein is an antibody.

32. The method of any one of claims 17-21, wherein the BH3 mimetic is an inhibitor of Bcl-2, Bcl-xL, Mcl-1, Bcl-W, A1/BFL-1, or a mixture thereof.

33. The method of claim 32, wherein the BH3 mimetic is a combination of an inhibitor of Bcl-2 and an inhibitor of Bcl-xL.

34. The method of claim 32, wherein the BH3 mimetic is a combination of an inhibitor of Bcl-2 and an inhibitor of Mcl-1.

35. The method of any one of claims 17-31, wherein the BH3 mimetic is not a Bcl-2 inhibitor.

36. The method of any one of claims 17-34, wherein the BH3 mimetic is AZD5991, ABT-737, ABT-199 (Venetoclax), ABT-263 (Navitoclax), or a mixture thereof.

37. The method of any one of claims 17-36, wherein the individual is greater than 1, 5, 10, 20, 30, 40, 50, or 60 years of age.

38. The method of claim 37, wherein the individual is greater than 60 years of age.

39. The method of any one of claims 17-38, further comprising the step of administering to the individual one or more activators of p53.

40. The method of claim 39, wherein the activator of p53 is Nutlin-3a, RG7112, RG7388, JNJ-26854165, MI-773, AMG 232, NVP-CGM097, HDM201, MK-8242, R06839921, DS-3032b, R05353, R02468, R08994, SAR405838, ALRN-6924 (MDM2/MDM4 dual antagonist), one or more MDM2 degraders, or a mixture thereof.

41. The method of claim 40, wherein the one or more MDM2 degraders is selected from the group consisting of LE-004, LE-102, LE-154, LE-157, LD-222, MD-224, and a combination thereof.

42. The method of any one of claims 17-41, further comprising the step of identifying the presence or level of p73 or an isoform thereof in a sample of cells from the individual of the individual.

43. A method of reducing or preventing cancer resistance to one or more BH3 mimetics in an individual, comprising the step of administering to the individual a therapeutically effective amount of one or more agents that target p73 or an isoform thereof.

44. The method of claim 43, wherein the p73 isoform is transactivation p73 (Tap73) or ΔNp73.

45. The method of claim 43 or 44, wherein the agent that targets p73 or an isoform thereof is a small molecule, protein, nucleic acid, or mixture or combination thereof.

46. The method of any one of claims 43-45, wherein the agent that targets p73 or an isoform thereof is octyl-(R)-2HG, octyl-(L)-2HG, or a mixture thereof..

47. The method of claim 45, wherein the protein is an antibody.

48. The method of claim 45, wherein the nucleic acid is shRNA or CRISPR.

49. The method of any one of claims 43-55, further comprising the step of administering a therapeutically effective amount of one or more BH3 mimetics to the individual.

50. The method of claim 49, wherein the BH3 mimetic is an inhibitor of Bcl-2, Bcl-xL, Mcl-1, Bcl-W, A1/BFL-1, or a combination thereof.

51. The method of claim 49 or 50, wherein the BH3 mimetic is a combination of an inhibitor of Bcl-2 and an inhibitor of Bcl-xL.

52. The method of claim 49 or 50, wherein the BH3 mimetic is a combination of an inhibitor of Bcl-2 and an inhibitor of Mcl-1.

53. The method of any one of claims 49-52, wherein the BH3 mimetic is AZD5991, ABT-737, ABT-199 (Venetoclax), ABT-263 (Navitoclax), or a mixture thereof.

54. The method of claim 49, wherein the BH3 mimetic is not a Bcl-2 inhibitor.

55. The method of any one of claims 43-54, wherein the individual has a hematological cancer or a solid tumor.

56. The method of claim 55, wherein the hematological cancer is chronic lymphocytic leukemia, acute myeloid leukemia, acute lymphocytic leukemia, chronic myeloid leukemia, Hodgkin's lymphoma, non-Hodgkin's lymphoma, or myeloma.

57. The method of any one of claims 43-56, further comprising the step of identifying the presence or level of p73 or an isoform thereof in a sample of cells from the individual.

58. The method of any one of claims 43-57, wherein the individual is greater than 1, 5, 10, 20, 30, 40, 50, or 60 years of age.

59. The method of claim 58, wherein the individual is greater than 60 years of age.

60. The method of any one of claims 43-59, further comprising the step of administering to the individual one or more activators of p53.

61. The method of claim 60, wherein the activator of p53 is Nutlin-3a, RG7112, RG7388, JNJ-26854165, MI-773, AMG 232, NVP-CGM097, HDM201, MK-8242, R06839921, DS-3032, APR-246, R05353, R02468, R08994, or a mixture thereof.

62. A kit comprising the composition of any one of claims 1-15.


SEQUENCE LISTING (24 sequences)

SEQ ID NO: 1 - DNA, 1764 bp, Homo sapiens
atgctgtacgtcggtgaccccgcacggcacctcgccacggcccagttcaatctgctgagcagcaccatggaccagatgagcagccgcgcggcctcggccagcccctacaccccagagcacgccgccagcgtgcccacccactcgccctacgcacaacccagctccaccttcgacaccatgtcgccggcgcctgtcatcccctccaacaccgactaccccggaccccaccactttgaggtcactttccagcagtccagcacggccaagtcagccacctggacgtactccccgctcttgaagaaactctactgccagatcgccaagacatgccccatccagatcaaggtgtccaccccgccacccccaggcaccgccatccgggccatgcctgtttacaagaaagcggagcacgtgaccgacgtcgtgaaacgctgccccaaccacgagctcgggagggacttcaacgaaggacagtctgctccagccagccacctcatccgcgtggaaggcaataatctctcgcagtatgtggatgaccctgtcaccggcaggcagagcgtcgtggtgccctatgagccaccacaggtggggacggaattcaccaccatcctgtacaacttcatgtgtaacagcagctgtgtagggggcatgaaccggcggcccatcctcatcatcatcaccctggagatgcgggatgggcaggtgctgggccgccggtcctttgagggccgcatctgcgcctgtcctggccgcgaccgaaaagctgatgaggaccactaccgggagcagcaggccctgaacgagagctccgccaagaacggggccgccagcaagcgtgccttcaagcagagcccccctgccgtccccgcccttggtgccggtgtgaagaagcggcggcatggagacgaggacacgtactaccttcaggtgcgaggccgggagaactttgagatcctgatgaagctgaaagagagcctggagctgatggagttggtgccgcagccactggtggactcctatcggcagcagcagcagctcctacagaggccgagtcacctacagcccccgtcctacgggccggtcctctcgcccatgaacaaggtgcacgggggcatgaacaagctgccctccgtcaaccagctggtgggccagcctcccccgcacagttcggcagctacacccaacctggggcccgtgggccccgggatgctcaacaaccatggccacgcagtgccagccaacggcgagatgagcagcagccacagcgcccagtccatggtctcggggtcccactgcactccgccacccccctaccacgccgaccccagcctcgtcagttttttaacaggattggggtgtccaaactgcatcgagtatttcacctcccaagggttacagagcatttaccacctgcagaacctgaccattgaggacctgggggccctgaagatccccgagcagtaccgcatgaccatctggcggggcctgcaggacctgaagcagggccacgactacagcaccgcgcagcagctgctccgctctagcaacgcggccaccatctccatcggcggctcaggggaactgcagcgccagcgggtcatggaggccgtgcacttccgcgtgcgccacaccatcaccatccccaaccgcggcggcccaggcggcggccctgacgagtgggcggacttcggcttcgacctgcccgactgcaaggcccgcaagcagcccatcaaggaggagttcacggaggccgagatccactga

SEQ ID NO: 2 - protein, 587 aa, Homo sapiens
MLYVGDPARHLATAQFNLLSSTMDQMSSRAASASPYTPEHAASVPTHSPYAQPSSTFDTMSPAPVIPSNTDYPGPHHFEVTFQQSSTAKSATWTYSPLLKKLYCQIAKTCPIQIKVSTPPPPGTAIRAMPVYKKAEHVTDVVKRCPNHELGRDFNEGQSAPASHLIRVEGNNLSQYVDDPVTGRQSVVVPYEPPQVGTEFTTILYNFMCNSSCVGGMNRRPILIIITLEMRDGQVLGRRSFEGRICACPGRDRKADEDHYREQQALNESSAKNGAASKRAFKQSPPAVPALGAGVKKRRHGDEDTYYLQVRGRENFEILMKLKESLELMELVPQPLVDSYRQQQQLLQRPSHLQPPSYGPVLSPMNKVHGGMNKLPSVNQLVGQPPPHSSAATPNLGPVGPGMLNNHGHAVPANGEMSSSHSAQSMVSGSHCTPPPPYHADPSLVSFLTGLGCPNCIEYFTSQGLQSIYHLQNLTIEDLGALKIPEQYRMTIWRGLQDLKQGHDYSTAQQLLRSSNAATISIGGSGELQRQRVMEAVHFRVRHTITIPNRGGPGGGPDEWADFGFDLPDCKARKQPIKEEFTEAEIH

SEQ ID NO: 3 - DNA, 1353 bp, Homo sapiens
atgctgtacgtcggtgaccccgcacggcacctcgccacggcccagttcaatctgctgagcagcaccatggaccagatgagcagccgcgcggcctcggccagcccctacaccccagagcacgccgccagcgtgcccacccactcgccctacgcacaacccagctccaccttcgacaccatgtcgccggcgcctgtcatcccctccaacaccgactaccccggaccccaccactttgaggtcactttccagcagtccagcacggccaagtcagccacctggacgtactccccgctcttgaagaaactctactgccagatcgccaagacatgccccatccagatcaaggtgtccaccccgccacccccaggcaccgccatccgggccatgcctgtttacaagaaagcggagcacgtgaccgacgtcgtgaaacgctgccccaaccacgagctcgggagggacttcaacgaaggacagtctgctccagccagccacctcatccgcgtggaaggcaataatctctcgcagtatgtggatgaccctgtcaccggcaggcagagcgtcgtggtgccctatgagccaccacaggtggggacggaattcaccaccatcctgtacaacttcatgtgtaacagcagctgtgtagggggcatgaaccggcggcccatcctcatcatcatcaccctggagatgcgggatgggcaggtgctgggccgccggtcctttgagggccgcatctgcgcctgtcctggccgcgaccgaaaagctgatgaggaccactaccgggagcagcaggccctgaacgagagctccgccaagaacggggccgccagcaagcgtgccttcaagcagagcccccctgccgtccccgcccttggtgccggtgtgaagaagcggcggcatggagacgaggacacgtactaccttcaggtgcgaggccgggagaactttgagatcctgatgaagctgaaagagagcctggagctgatggagttggtgccgcagccactggtggactcctatcggcagcagcagcagctcctacagaggccgagtcacctacagcccccgtcctacgggccggtcctctcgcccatgaacaaggtgcacgggggcatgaacaagctgccctccgtcaaccagctggtgggccagcctcccccgcacagttcggcagctacacccaacctggggcccgtgggccccgggatgctcaacaaccatggccacgcagtgccagccaacggcgagatgagcagcagccacagcgcccagtccatggtctcggggtcccactgcactccgccacccccctaccacgccgaccccagcctcgtcaggacctgggggccctga

SEQ ID NO: 4 - protein, 450 aa, Homo sapiens
MLYVGDPARHLATAQFNLLSSTMDQMSSRAASASPYTPEHAASVPTHSPYAQPSSTFDTMSPAPVIPSNTDYPGPHHFEVTFQQSSTAKSATWTYSPLLKKLYCQIAKTCPIQIKVSTPPPPGTAIRAMPVYKKAEHVTDVVKRCPNHELGRDFNEGQSAPASHLIRVEGNNLSQYVDDPVTGRQSVVVPYEPPQVGTEFTTILYNFMCNSSCVGGMNRRPILIIITLEMRDGQVLGRRSFEGRICACPGRDRKADEDHYREQQALNESSAKNGAASKRAFKQSPPAVPALGAGVKKRRHGDEDTYYLQVRGRENFEILMKLKESLELMELVPQPLVDSYRQQQQLLQRPSHLQPPSYGPVLSPMNKVHGGMNKLPSVNQLVGQPPPHSSAATPNLGPVGPGMLNNHGHAVPANGEMSSSHSAQSMVSGSHCTPPPPYHADPSLVRTWGP

SEQ ID NO: 5 - DNA, 1281 bp, Homo sapiens
atgctgtacgtcggtgaccccgcacggcacctcgccacggcccagttcaatctgctgagcagcaccatggaccagatgagcagccgcgcggcctcggccagcccctacaccccagagcacgccgccagcgtgcccacccactcgccctacgcacaacccagctccaccttcgacaccatgtcgccggcgcctgtcatcccctccaacaccgactaccccggaccccaccactttgaggtcactttccagcagtccagcacggccaagtcagccacctggacgtactccccgctcttgaagaaactctactgccagatcgccaagacatgccccatccagatcaaggtgtccaccccgccacccccaggcaccgccatccgggccatgcctgtttacaagaaagcggagcacgtgaccgacgtcgtgaaacgctgccccaaccacgagctcgggagggacttcaacgaaggacagtctgctccagccagccacctcatccgcgtggaaggcaataatctctcgcagtatgtggatgaccctgtcaccggcaggcagagcgtcgtggtgccctatgagccaccacaggtggggacggaattcaccaccatcctgtacaacttcatgtgtaacagcagctgtgtagggggcatgaaccggcggcccatcctcatcatcatcaccctggagatgcgggatgggcaggtgctgggccgccggtcctttgagggccgcatctgcgcctgtcctggccgcgaccgaaaagctgatgaggaccactaccgggagcagcaggccctgaacgagagctccgccaagaacggggccgccagcaagcgtgccttcaagcagagcccccctgccgtccccgcccttggtgccggtgtgaagaagcggcggcatggagacgaggacacgtactaccttcaggtgcgaggccgggagaactttgagatcctgatgaagctgaaagagagcctggagctgatggagttggtgccgcagccactggtggactcctatcggcagcagcagcagctcctacagaggccgccccgggatgctcaacaaccatggccacgcagtgccagccaacggcgagatgagcagcagccacagcgcccagtccatggtctcggggtcccactgcactccgccacccccctaccacgccgaccccagcctcgtcagttttttaacaggattggggtgtccaaactgcatcgagtatttcacctcccaagggttacagagcatttaccacctgcagaacctgaccattga

SEQ ID NO: 6 - protein, 426 aa, Homo sapiens
MLYVGDPARHLATAQFNLLSSTMDQMSSRAASASPYTPEHAASVPTHSPYAQPSSTFDTMSPAPVIPSNTDYPGPHHFEVTFQQSSTAKSATWTYSPLLKKLYCQIAKTCPIQIKVSTPPPPGTAIRAMPVYKKAEHVTDVVKRCPNHELGRDFNEGQSAPASHLIRVEGNNLSQYVDDPVTGRQSVVVPYEPPQVGTEFTTILYNFMCNSSCVGGMNRRPILIIITLEMRDGQVLGRRSFEGRICACPGRDRKADEDHYREQQALNESSAKNGAASKRAFKQSPPAVPALGAGVKKRRHGDEDTYYLQVRGRENFEILMKLKESLELMELVPQPLVDSYRQQQQLLQRPPRDAQQPWPRSASQRRDEQQPQRPVHGLGVPLHSATPLPRRPQPRQFFNRIGVSKLHRVFHLPRVTEHLPPAEPDH

SEQ ID NO: 7 - DNA, 1065 bp, Homo sapiens
atgctgtacgtcggtgaccccgcacggcacctcgccacggcccagttcaatctgctgagcagcaccatggaccagatgagcagccgcgcggcctcggccagcccctacaccccagagcacgccgccagcgtgcccacccactcgccctacgcacaacccagctccaccttcgacaccatgtcgccggcgcctgtcatcccctccaacaccgactaccccggaccccaccactttgaggtcactttccagcagtccagcacggccaagtcagccacctggacgtactccccgctcttgaagaaactctactgccagatcgccaagacatgccccatccagatcaaggtgtccaccccgccacccccaggcaccgccatccgggccatgcctgtttacaagaaagcggagcacgtgaccgacgtcgtgaaacgctgccccaaccacgagctcgggagggacttcaacgaaggacagtctgctccagccagccacctcatccgcgtggaaggcaataatctctcgcagtatgtggatgaccctgtcaccggcaggcagagcgtcgtggtgccctatgagccaccacaggtggggacggaattcaccaccatcctgtacaacttcatgtgtaacagcagctgtgtagggggcatgaaccggcggcccatcctcatcatcatcaccctggagatgcgggatgggcaggtgctgggccgccggtcctttgagggccgcatctgcgcctgtcctggccgcgaccgaaaagctgatgaggaccactaccgggagcagcaggccctgaacgagagctccgccaagaacggggccgccagcaagcgtgccttcaagcagagcccccctgccgtccccgcccttggtgccggtgtgaagaagcggcggcatggagacgaggacacgtactaccttcaggtgcgaggccgggagaactttgagatcctgatgaagctgaaagagagcctggagctgatggagttggtgccgcagccactggtggactcctatcggcagcagcagcagctcctacagaggccgacctgggggccctga

SEQ ID NO: 8 - protein, 354 aa, Homo sapiens
MLYVGDPARHLATAQFNLLSSTMDQMSSRAASASPYTPEHAASVPTHSPYAQPSSTFDTMSPAPVIPSNTDYPGPHHFEVTFQQSSTAKSATWTYSPLLKKLYCQIAKTCPIQIKVSTPPPPGTAIRAMPVYKKAEHVTDVVKRCPNHELGRDFNEGQSAPASHLIRVEGNNLSQYVDDPVTGRQSVVVPYEPPQVGTEFTTILYNFMCNSSCVGGMNRRPILIIITLEMRDGQVLGRRSFEGRICACPGRDRKADEDHYREQQALNESSAKNGAASKRAFKQSPPAVPALGAGVKKRRHGDEDTYYLQVRGRENFEILMKLKESLELMELVPQPLVDSYRQQQQLLQRPTWGP

SEQ ID NO: 9 - DNA, 1521 bp, Homo sapiens
atgctgtacgtcggtgaccccgcacggcacctcgccacggcccagttcaatctgctgagcagcaccatggaccagatgagcagccgcgcggcctcggccagcccctacaccccagagcacgccgccagcgtgcccacccactcgccctacgcacaacccagctccaccttcgacaccatgtcgccggcgcctgtcatcccctccaacaccgactaccccggaccccaccactttgaggtcactttccagcagtccagcacggccaagtcagccacctggacgtactccccgctcttgaagaaactctactgccagatcgccaagacatgccccatccagatcaaggtgtccaccccgccacccccaggcaccgccatccgggccatgcctgtttacaagaaagcggagcacgtgaccgacgtcgtgaaacgctgccccaaccacgagctcgggagggacttcaacgaaggacagtctgctccagccagccacctcatccgcgtggaaggcaataatctctcgcagtatgtggatgaccctgtcaccggcaggcagagcgtcgtggtgccctatgagccaccacaggtggggacggaattcaccaccatcctgtacaacttcatgtgtaacagcagctgtgtagggggcatgaaccggcggcccatcctcatcatcatcaccctggagatgcgggatgggcaggtgctgggccgccggtcctttgagggccgcatctgcgcctgtcctggccgcgaccgaaaagctgatgaggaccactaccgggagcagcaggccctgaacgagagctccgccaagaacggggccgccagcaagcgtgccttcaagcagagcccccctgccgtccccgcccttggtgccggtgtgaagaagcggcggcatggagacgaggacacgtactaccttcaggtgcgaggccgggagaactttgagatcctgatgaagctgaaagagagcctggagctgatggagttggtgccgcagccactggtggactcctatcggcagcagcagcagctcctacagaggccgccccgggatgctcaacaaccatggccacgcagtgccagccaacggcgagatgagcagcagccacagcgcccagtccatggtctcggggtcccactgcactccgccacccccctaccacgccgaccccagcctcgtcaggacctgggggccctgaagatccccgagcagtaccgcatgaccatctggcggggcctgcaggacctgaagcagggccacgactacagcaccgcgcagcagctgctccgctctagcaacgcggccaccatctccatcggcggctcaggggaactgcagcgccagcgggtcatggaggccgtgcacttccgcgtgcgccacaccatcaccatccccaaccgcggcggcccaggcggcggccctgacgagtgggcggacttcggcttcgacctgcccgactgcaaggcccgcaagcagcccatcaaggaggagttcacggaggccgagatccactga

SEQ ID NO: 10 - protein, 506 aa, Homo sapiens
MLYVGDPARHLATAQFNLLSSTMDQMSSRAASASPYTPEHAASVPTHSPYAQPSSTFDTMSPAPVIPSNTDYPGPHHFEVTFQQSSTAKSATWTYSPLLKKLYCQIAKTCPIQIKVSTPPPPGTAIRAMPVYKKAEHVTDVVKRCPNHELGRDFNEGQSAPASHLIRVEGNNLSQYVDDPVTGRQSVVVPYEPPQVGTEFTTILYNFMCNSSCVGGMNRRPILIIITLEMRDGQVLGRRSFEGRICACPGRDRKADEDHYREQQALNESSAKNGAASKRAFKQSPPAVPALGAGVKKRRHGDEDTYYLQVRGRENFEILMKLKESLELMELVPQPLVDSYRQQQQLLQRPPRDAQQPWPRSASQRRDEQQPQRPVHGLGVPLHSATPLPRRPQPRQDLGALKIPEQYRMTIWRGLQDLKQGHDYSTAQQLLRSSNAATISIGGSGELQRQRVMEAVHFRVRHTITIPNRGGPGGGPDEWADFGFDLPDCKARKQPIKEEFTEAEIH

SEQ ID NO: 11 - DNA, 1476 bp, Homo sapiens
atgctgtacgtcggtgaccccgcacggcacctcgccacggcccagttcaatctgctgagcagcaccatggaccagatgagcagccgcgcggcctcggccagcccctacaccccagagcacgccgccagcgtgcccacccactcgccctacgcacaacccagctccaccttcgacaccatgtcgccggcgcctgtcatcccctccaacaccgactaccccggaccccaccactttgaggtcactttccagcagtccagcacggccaagtcagccacctggacgtactccccgctcttgaagaaactctactgccagatcgccaagacatgccccatccagatcaaggtgtccaccccgccacccccaggcaccgccatccgggccatgcctgtttacaagaaagcggagcacgtgaccgacgtcgtgaaacgctgccccaaccacgagctcgggagggacttcaacgaaggacagtctgctccagccagccacctcatccgcgtggaaggcaataatctctcgcagtatgtggatgaccctgtcaccggcaggcagagcgtcgtggtgccctatgagccaccacaggtggggacggaattcaccaccatcctgtacaacttcatgtgtaacagcagctgtgtagggggcatgaaccggcggcccatcctcatcatcatcaccctggagatgcgggatgggcaggtgctgggccgccggtcctttgagggccgcatctgcgcctgtcctggccgcgaccgaaaagctgatgaggaccactaccgggagcagcaggccctgaacgagagctccgccaagaacggggccgccagcaagcgtgccttcaagcagagcccccctgccgtccccgcccttggtgccggtgtgaagaagcggcggcatggagacgaggacacgtactaccttcaggtgcgaggccgggagaactttgagatcctgatgaagctgaaagagagcctggagctgatggagttggtgccgcagccactggtggactcctatcggcagcagcagcagctcctacagaggccttttttaacaggattggggtgtccaaactgcatcgagtatttcacctcccaagggttacagagcatttaccacctgcagaacctgaccattgaggacctgggggccctgaagatccccgagcagtaccgcatgaccatctggcggggcctgcaggacctgaagcagggccacgactacagcaccgcgcagcagctgctccgctctagcaacgcggccaccatctccatcggcggctcaggggaactgcagcgccagcgggtcatggaggccgtgcacttccgcgtgcgccacaccatcaccatccccaaccgcggcggcccaggcggcggccctgacgagtgggcggacttcggcttcgacctgcccgactgcaaggcccgcaagcagcccatcaaggaggagttcacggaggccgagatccactga

SEQ ID NO: 12 - protein, 491 aa, Homo sapiens
MLYVGDPARHLATAQFNLLSSTMDQMSSRAASASPYTPEHAASVPTHSPYAQPSSTFDTMSPAPVIPSNTDYPGPHHFEVTFQQSSTAKSATWTYSPLLKKLYCQIAKTCPIQIKVSTPPPPGTAIRAMPVYKKAEHVTDVVKRCPNHELGRDFNEGQSAPASHLIRVEGNNLSQYVDDPVTGRQSVVVPYEPPQVGTEFTTILYNFMCNSSCVGGMNRRPILIIITLEMRDGQVLGRRSFEGRICACPGRDRKADEDHYREQQALNESSAKNGAASKRAFKQSPPAVPALGAGVKKRRHGDEDTYYLQVRGRENFEILMKLKESLELMELVPQPLVDSYRQQQQLLQRPFLTGLGCPNCIEYFTSQGLQSIYHLQNLTIEDLGALKIPEQYRMTIWRGLQDLKQGHDYSTAQQLLRSSNAATISIGGSGELQRQRVMEAVHFRVRHTITIPNRGGPGGGPDEWADFGFDLPDCKARKQPIKEEFTEAEIH

SEQ ID NO: 13 - DNA, 1911 bp, Homo sapiens
atggcccagtccaccgccacctcccctgatgggggcaccacgtttgagcacctctggagctctctggaaccagacagcacctacttcgaccttccccagtcaagccgggggaataatgaggtggtgggcggaacggattccagcatggacgtcttccacctggagggcatgactacatctgtcatggcccagttcaatctgctgagcagcaccatggaccagatgagcagccgcgcggcctcggccagcccctacaccccagagcacgccgccagcgtgcccacccactcgccctacgcacaacccagctccaccttcgacaccatgtcgccggcgcctgtcatcccctccaacaccgactaccccggaccccaccactttgaggtcactttccagcagtccagcacggccaagtcagccacctggacgtactccccgctcttgaagaaactctactgccagatcgccaagacatgccccatccagatcaaggtgtccaccccgccacccccaggcaccgccatccgggccatgcctgtttacaagaaagcggagcacgtgaccgacgtcgtgaaacgctgccccaaccacgagctcgggagggacttcaacgaaggacagtctgctccagccagccacctcatccgcgtggaaggcaataatctctcgcagtatgtggatgaccctgtcaccggcaggcagagcgtcgtggtgccctatgagccaccacaggtggggacggaattcaccaccatcctgtacaacttcatgtgtaacagcagctgtgtagggggcatgaaccggcggcccatcctcatcatcatcaccctggagatgcgggatgggcaggtgctgggccgccggtcctttgagggccgcatctgcgcctgtcctggccgcgaccgaaaagctgatgaggaccactaccgggagcagcaggccctgaacgagagctccgccaagaacggggccgccagcaagcgtgccttcaagcagagcccccctgccgtccccgcccttggtgccggtgtgaagaagcggcggcatggagacgaggacacgtactaccttcaggtgcgaggccgggagaactttgagatcctgatgaagctgaaagagagcctggagctgatggagttggtgccgcagccactggtggactcctatcggcagcagcagcagctcctacagaggccgagtcacctacagcccccgtcctacgggccggtcctctcgcccatgaacaaggtgcacgggggcatgaacaagctgccctccgtcaaccagctggtgggccagcctcccccgcacagttcggcagctacacccaacctggggcccgtgggccccgggatgctcaacaaccatggccacgcagtgccagccaacggcgagatgagcagcagccacagcgcccagtccatggtctcggggtcccactgcactccgccacccccctaccacgccgaccccagcctcgtcagttttttaacaggattggggtgtccaaactgcatcgagtatttcacctcccaagggttacagagcatttaccacctgcagaacctgaccattgaggacctgggggccctgaagatccccgagcagtaccgcatgaccatctggcggggcctgcaggacctgaagcagggccacgactacagcaccgcgcagcagctgctccgctctagcaacgcggccaccatctccatcggcggctcaggggaactgcagcgccagcgggtcatggaggccgtgcacttccgcgtgcgccacaccatcaccatccccaaccgcggcggcccaggcggcggccctgacgagtgggcggacttcggcttcgacctgcccgactgcaaggcccgcaagcagcccatcaaggaggagttcacggaggccgagatccactga

SEQ ID NO: 14 - protein, 636 aa, Homo sapiens
MAQSTATSPDGGTTFEHLWSSLEPDSTYFDLPQSSRGNNEVVGGTDSSMDVFHLEGMTTSVMAQFNLLSSTMDQMSSRAASASPYTPEHAASVPTHSPYAQPSSTFDTMSPAPVIPSNTDYPGPHHFEVTFQQSSTAKSATWTYSPLLKKLYCQIAKTCPIQIKVSTPPPPGTAIRAMPVYKKAEHVTDVVKRCPNHELGRDFNEGQSAPASHLIRVEGNNLSQYVDDPVTGRQSVVVPYEPPQVGTEFTTILYNFMCNSSCVGGMNRRPILIIITLEMRDGQVLGRRSFEGRICACPGRDRKADEDHYREQQALNESSAKNGAASKRAFKQSPPAVPALGAGVKKRRHGDEDTYYLQVRGRENFEILMKLKESLELMELVPQPLVDSYRQQQQLLQRPSHLQPPSYGPVLSPMNKVHGGMNKLPSVNQLVGQPPPHSSAATPNLGPVGPGMLNNHGHAVPANGEMSSSHSAQSMVSGSHCTPPPPYHADPSLVSFLTGLGCPNCIEYFTSQGLQSIYHLQNLTIEDLGALKIPEQYRMTIWRGLQDLKQGHDYSTAQQLLRSSNAATISIGGSGELQRQRVMEAVHFRVRHTITIPNRGGPGGGPDEWADFGFDLPDCKARKQPIKEEFTEAEIH

SEQ ID NO: 15 - DNA, 1500 bp, Homo sapiens
atggcccagtccaccgccacctcccctgatgggggcaccacgtttgagcacctctggagctctctggaaccagacagcacctacttcgaccttccccagtcaagccgggggaataatgaggtggtgggcggaacggattccagcatggacgtcttccacctggagggcatgactacatctgtcatggcccagttcaatctgctgagcagcaccatggaccagatgagcagccgcgcggcctcggccagcccctacaccccagagcacgccgccagcgtgcccacccactcgccctacgcacaacccagctccaccttcgacaccatgtcgccggcgcctgtcatcccctccaacaccgactaccccggaccccaccactttgaggtcactttccagcagtccagcacggccaagtcagccacctggacgtactccccgctcttgaagaaactctactgccagatcgccaagacatgccccatccagatcaaggtgtccaccccgccacccccaggcaccgccatccgggccatgcctgtttacaagaaagcggagcacgtgaccgacgtcgtgaaacgctgccccaaccacgagctcgggagggacttcaacgaaggacagtctgctccagccagccacctcatccgcgtggaaggcaataatctctcgcagtatgtggatgaccctgtcaccggcaggcagagcgtcgtggtgccctatgagccaccacaggtggggacggaattcaccaccatcctgtacaacttcatgtgtaacagcagctgtgtagggggcatgaaccggcggcccatcctcatcatcatcaccctggagatgcgggatgggcaggtgctgggccgccggtcctttgagggccgcatctgcgcctgtcctggccgcgaccgaaaagctgatgaggaccactaccgggagcagcaggccctgaacgagagctccgccaagaacggggccgccagcaagcgtgccttcaagcagagcccccctgccgtccccgcccttggtgccggtgtgaagaagcggcggcatggagacgaggacacgtactaccttcaggtgcgaggccgggagaactttgagatcctgatgaagctgaaagagagcctggagctgatggagttggtgccgcagccactggtggactcctatcggcagcagcagcagctcctacagaggccgagtcacctacagcccccgtcctacgggccggtcctctcgcccatgaacaaggtgcacgggggcatgaacaagctgccctccgtcaaccagctggtgggccagcctcccccgcacagttcggcagctacacccaacctggggcccgtgggccccgggatgctcaacaaccatggccacgcagtgccagccaacggcgagatgagcagcagccacagcgcccagtccatggtctcggggtcccactgcactccgccacccccctaccacgccgaccccagcctcgtcaggacctgggggccctga

SEQ ID NO: 16 - protein, 499 aa, Homo sapiens
MAQSTATSPDGGTTFEHLWSSLEPDSTYFDLPQSSRGNNEVVGGTDSSMDVFHLEGMTTSVMAQFNLLSSTMDQMSSRAASASPYTPEHAASVPTHSPYAQPSSTFDTMSPAPVIPSNTDYPGPHHFEVTFQQSSTAKSATWTYSPLLKKLYCQIAKTCPIQIKVSTPPPPGTAIRAMPVYKKAEHVTDVVKRCPNHELGRDFNEGQSAPASHLIRVEGNNLSQYVDDPVTGRQSVVVPYEPPQVGTEFTTILYNFMCNSSCVGGMNRRPILIIITLEMRDGQVLGRRSFEGRICACPGRDRKADEDHYREQQALNESSAKNGAASKRAFKQSPPAVPALGAGVKKRRHGDEDTYYLQVRGRENFEILMKLKESLELMELVPQPLVDSYRQQQQLLQRPSHLQPPSYGPVLSPMNKVHGGMNKLPSVNQLVGQPPPHSSAATPNLGPVGPGMLNNHGHAVPANGEMSSSHSAQSMVSGSHCTPPPPYHADPSLVRTWGP

SEQ ID NO: 17 - DNA, 1428 bp, Homo sapiens
atggcccagtccaccgccacctcccctgatgggggcaccacgtttgagcacctctggagctctctggaaccagacagcacctacttcgaccttccccagtcaagccgggggaataatgaggtggtgggcggaacggattccagcatggacgtcttccacctggagggcatgactacatctgtcatggcccagttcaatctgctgagcagcaccatggaccagatgagcagccgcgcggcctcggccagcccctacaccccagagcacgccgccagcgtgcccacccactcgccctacgcacaacccagctccaccttcgacaccatgtcgccggcgcctgtcatcccctccaacaccgactaccccggaccccaccactttgaggtcactttccagcagtccagcacggccaagtcagccacctggacgtactccccgctcttgaagaaactctactgccagatcgccaagacatgccccatccagatcaaggtgtccaccccgccacccccaggcaccgccatccgggccatgcctgtttacaagaaagcggagcacgtgaccgacgtcgtgaaacgctgccccaaccacgagctcgggagggacttcaacgaaggacagtctgctccagccagccacctcatccgcgtggaaggcaataatctctcgcagtatgtggatgaccctgtcaccggcaggcagagcgtcgtggtgccctatgagccaccacaggtggggacggaattcaccaccatcctgtacaacttcatgtgtaacagcagctgtgtagggggcatgaaccggcggcccatcctcatcatcatcaccctggagatgcgggatgggcaggtgctgggccgccggtcctttgagggccgcatctgcgcctgtcctggccgcgaccgaaaagctgatgaggaccactaccgggagcagcaggccctgaacgagagctccgccaagaacggggccgccagcaagcgtgccttcaagcagagcccccctgccgtccccgcccttggtgccggtgtgaagaagcggcggcatggagacgaggacacgtactaccttcaggtgcgaggccgggagaactttgagatcctgatgaagctgaaagagagcctggagctgatggagttggtgccgcagccactggtggactcctatcggcagcagcagcagctcctacagaggccgccccgggatgctcaacaaccatggccacgcagtgccagccaacggcgagatgagcagcagccacagcgcccagtccatggtctcggggtcccactgcactccgccacccccctaccacgccgaccccagcctcgtcagttttttaacaggattggggtgtccaaactgcatcgagtatttcacctcccaagggttacagagcatttaccacctgcagaacctgaccattga

SEQ ID NO: 18 - protein, 476 aa, Homo sapiens
MAQSTATSPDGGTTFEHLWSSLEPDSTYFDLPQSSRGNNEVVGGTDSSMDVFHLEGMTTSVMAQFNLLSSTMDQMSSRAASASPYTPEHAASVPTHSPYAQPSSTFDTMSPAPVIPSNTDYPGPHHFEVTFQQSSTAKSATWTYSPLLKKLYCQIAKTCPIQIKVSTPPPPGTAIRAMPVYKKAEHVTDVVKRCPNHELGRDFNEGQSAPASHLIRVEGNNLSQYVDDPVTGRQSVVVPYEPPQVGTEFTTILYNFMCNSSCVGGMNRRPILIIITLEMRDGQVLGRRSFEGRICACPGRDRKADEDHYREQQALNESSAKNGAASKRAFKQSPPAVPALGAGVKKRRHGDEDTYYLQVRGRENFEILMKLKESLELMELVPQPLVDSYRQQQQLLQRPPRDAQQPWPRSASQQRRDEQQPQRPVHGLGVPLHSATPLPRRPQPRQFFNRIGVSKLHRVFHLPRVTEHLPPAEPDH

SEQ ID NO: 19 - DNA, 1212 bp, Homo sapiens
atggcccagtccaccgccacctcccctgatgggggcaccacgtttgagcacctctggagctctctggaaccagacagcacctacttcgaccttccccagtcaagccgggggaataatgaggtggtgggcggaacggattccagcatggacgtcttccacctggagggcatgactacatctgtcatggcccagttcaatctgctgagcagcaccatggaccagatgagcagccgcgcggcctcggccagcccctacaccccagagcacgccgccagcgtgcccacccactcgccctacgcacaacccagctccaccttcgacaccatgtcgccggcgcctgtcatcccctccaacaccgactaccccggaccccaccactttgaggtcactttccagcagtccagcacggccaagtcagccacctggacgtactccccgctcttgaagaaactctactgccagatcgccaagacatgccccatccagatcaaggtgtccaccccgccacccccaggcaccgccatccgggccatgcctgtttacaagaaagcggagcacgtgaccgacgtcgtgaaacgctgccccaaccacgagctcgggagggacttcaacgaaggacagtctgctccagccagccacctcatccgcgtggaaggcaataatctctcgcagtatgtggatgaccctgtcaccggcaggcagagcgtcgtggtgccctatgagccaccacaggtggggacggaattcaccaccatcctgtacaacttcatgtgtaacagcagctgtgtagggggcatgaaccggcggcccatcctcatcatcatcaccctggagatgcgggatgggcaggtgctgggccgccggtcctttgagggccgcatctgcgcctgtcctggccgcgaccgaaaagctgatgaggaccactaccgggagcagcaggccctgaacgagagctccgccaagaacggggccgccagcaagcgtgccttcaagcagagcccccctgccgtccccgcccttggtgccggtgtgaagaagcggcggcatggagacgaggacacgtactaccttcaggtgcgaggccgggagaactttgagatcctgatgaagctgaaagagagcctggagctgatggagttggtgccgcagccactggtggactcctatcggcagcagcagcagctcctacagaggccgacctgggggccctga

SEQ ID NO: 20 - protein, 403 aa, Homo sapiens
MAQSTATSPDGGTTFEHLWSSLEPDSTYFDLPQSSRGNNEVVGGTDSSMDVFHLEGMTTSVMAQFNLLSSTMDQMSSRAASASPYTPEHAASVPTHSPYAQPSSTFDTMSPAPVIPSNTDYPGPHHFEVTFQQSSTAKSATWTYSPLLKKLYCQIAKTCPIQIKVSTPPPPGTAIRAMPVYKKAEHVTDVVKRCPNHELGRDFNEGQSAPASHLIRVEGNNLSQYVDDPVTGRQSVVVPYEPPQVGTEFTTILYNFMCNSSCVGGMNRRPILIIITLEMRDGQVLGRRSFEGRICACPGRDRKADEDHYREQQALNESSAKNGAASKRAFKQSPPAVPALGAGVKKRRHGDEDTYYLQVRGRENFEILMKLKESLELMELVPQPLVDSYRQQQQLLQRPTWGP

SEQ ID NO: 21 - DNA, 1668 bp, Homo sapiens
atggcccagtccaccgccacctcccctgatgggggcaccacgtttgagcacctctggagctctctggaaccagacagcacctacttcgaccttccccagtcaagccgggggaataatgaggtggtgggcggaacggattccagcatggacgtcttccacctggagggcatgactacatctgtcatggcccagttcaatctgctgagcagcaccatggaccagatgagcagccgcgcggcctcggccagcccctacaccccagagcacgccgccagcgtgcccacccactcgccctacgcacaacccagctccaccttcgacaccatgtcgccggcgcctgtcatcccctccaacaccgactaccccggaccccaccactttgaggtcactttccagcagtccagcacggccaagtcagccacctggacgtactccccgctcttgaagaaactctactgccagatcgccaagacatgccccatccagatcaaggtgtccaccccgccacccccaggcaccgccatccgggccatgcctgtttacaagaaagcggagcacgtgaccgacgtcgtgaaacgctgccccaaccacgagctcgggagggacttcaacgaaggacagtctgctccagccagccacctcatccgcgtggaaggcaataatctctcgcagtatgtggatgaccctgtcaccggcaggcagagcgtcgtggtgccctatgagccaccacaggtggggacggaattcaccaccatcctgtacaacttcatgtgtaacagcagctgtgtagggggcatgaaccggcggcccatcctcatcatcatcaccctggagatgcgggatgggcaggtgctgggccgccggtcctttgagggccgcatctgcgcctgtcctggccgcgaccgaaaagctgatgaggaccactaccgggagcagcaggccctgaacgagagctccgccaagaacggggccgccagcaagcgtgccttcaagcagagcccccctgccgtccccgcccttggtgccggtgtgaagaagcggcggcatggagacgaggacacgtactaccttcaggtgcgaggccgggagaactttgagatcctgatgaagctgaaagagagcctggagctgatggagttggtgccgcagccactggtggactcctatcggcagcagcagcagctcctacagaggccgccccgggatgctcaacaaccatggccacgcagtgccagccaacggcgagatgagcagcagccacagcgcccagtccatggtctcggggtcccactgcactccgccacccccctaccacgccgaccccagcctcgtcaggacctgggggccctgaagatccccgagcagtaccgcatgaccatctggcggggcctgcaggacctgaagcagggccacgactacagcaccgcgcagcagctgctccgctctagcaacgcggccaccatctccatcggcggctcaggggaactgcagcgccagcgggtcatggaggccgtgcacttccgcgtgcgccacaccatcaccatccccaaccgcggcggcccaggcggcggccctgacgagtgggcggacttcggcttcgacctgcccgactgcaaggcccgcaagcagcccatcaaggaggagttcacggaggccgagatccactga

SEQ ID NO: 22 - protein, 555 aa, Homo sapiens
MAQSTATSPDGGTTFEHLWSSLEPDSTYFDLPQSSRGNNEVVGGTDSSMDVFHLEGMTTSVMAQFNLLSSTMDQMSSRAASASPYTPEHAASVPTHSPYAQPSSTFDTMSPAPVIPSNTDYPGPHHFEVTFQQSSTAKSATWTYSPLLKKLYCQIAKTCPIQIKVSTPPPPGTAIRAMPVYKKAEHVTDVVKRCPNHELGRDFNEGQSAPASHLIRVEGNNLSQYVDDPVTGRQSVVVPYEPPQVGTEFTTILYNFMCNSSCVGGMNRRPILIIITLEMRDGQVLGRRSFEGRICACPGRDRKADEDHYREQQALNESSAKNGAASKRAFKQSPPAVPALGAGVKKRRHGDEDTYYLQVRGRENFEILMKLKESLELMELVPQPLVDSYRQQQQLLQRPPRDAQQPWPRSASQQRRDEQQPQRPVHGLGVPLHSATPLPRRPQPRDLGALKIPEQYRMTIWRGLQDLKQGHDYSTAQQLLRSSNAATISIGGSGELQRQRVMEAVHFRVRHTITIPNRGGPGGGPDEWADFGFDLPDCKARKQPIKEEFTEAEIH

SEQ ID NO: 23 - DNA, 1623 bp, Homo sapiens
atggcccagtccaccgccacctcccctgatgggggcaccacgtttgagcacctctggagctctctggaaccagacagcacctacttcgaccttccccagtcaagccgggggaataatgaggtggtgggcggaacggattccagcatggacgtcttccacctggagggcatgactacatctgtcatggcccagttcaatctgctgagcagcaccatggaccagatgagcagccgcgcggcctcggccagcccctacaccccagagcacgccgccagcgtgcccacccactcgccctacgcacaacccagctccaccttcgacaccatgtcgccggcgcctgtcatcccctccaacaccgactaccccggaccccaccactttgaggtcactttccagcagtccagcacggccaagtcagccacctggacgtactccccgctcttgaagaaactctactgccagatcgccaagacatgccccatccagatcaaggtgtccaccccgccacccccaggcaccgccatccgggccatgcctgtttacaagaaagcggagcacgtgaccgacgtcgtgaaacgctgccccaaccacgagctcgggagggacttcaacgaaggacagtctgctccagccagccacctcatccgcgtggaaggcaataatctctcgcagtatgtggatgaccctgtcaccggcaggcagagcgtcgtggtgccctatgagccaccacaggtggggacggaattcaccaccatcctgtacaacttcatgtgtaacagcagctgtgtagggggcatgaaccggcggcccatcctcatcatcatcaccctggagatgcgggatgggcaggtgctgggccgccggtcctttgagggccgcatctgcgcctgtcctggccgcgaccgaaaagctgatgaggaccactaccgggagcagcaggccctgaacgagagctccgccaagaacggggccgccagcaagcgtgccttcaagcagagcccccctgccgtccccgcccttggtgccggtgtgaagaagcggcggcatggagacgaggacacgtactaccttcaggtgcgaggccgggagaactttgagatcctgatgaagctgaaagagagcctggagctgatggagttggtgccgcagccactggtggactcctatcggcagcagcagcagctcctacagaggccttttttaacaggattggggtgtccaaactgcatcgagtatttcacctcccaagggttacagagcatttaccacctgcagaacctgaccattgaggacctgggggccctgaagatccccgagcagtaccgcatgaccatctggcggggcctgcaggacctgaagcagggccacgactacagcaccgcgcagcagctgctccgctctagcaacgcggccaccatctccatcggcggctcaggggaactgcagcgccagcgggtcatggaggccgtgcacttccgcgtgcgccacaccatcaccatccccaaccgcggcggcccaggcggcggccctgacgagtgggcggacttcggcttcgacctgcccgactgcaaggcccgcaagcagcccatcaaggaggagttcacggaggccgagatccactga

SEQ ID NO: 24 - protein, 540 aa, Homo sapiens
MAQSTATSPDGGTTFEHLWSSLEPDSTYFDLPQSSRGNNEVVGGTDSSMDVFHLEGMTTSVMAQFNLLSSTMDQMSSRAASASPYTPEHAASVPTHSPYAQPSSTFDTMSPAPVIPSNTDYPGPHHFEVTFQQSSTAKSATWTYSPLLKKLYCQIAKTCPIQIKVSTPPPPGTAIRAMPVYKKAEHVTDVVKRCPNHELGRDFNEGQSAPASHLIRVEGNNLSQYVDDPVTGRQSVVVPYEPPQVGTEFTTILYNFMCNSSCVGGMNRRPILIIITLEMRDGQVLGRRSFEGRICACPGRDRKADEDHYREQQALNESSAKNGAASKRAFKQSPPAVPALGAGVKKRRHGDEDTYYLQVRGRENFEILMKLKESLELMELVPQPLVDSYRQQQQLLQRPFLTGLGCPNCIEYFTSQGLQSIYHLQNLTIEDLGALKIPEQYRMTIWRGLQDLKQGHDYSTAQQLLRSSNAATISIGGSGELQRQRVMEAVHFRVRHTITIPNRGGPGGGPDEWADFGFDLPDCKARKQPIKEEFTEAEIH